(19) United States
(12) Patent Application Publication
(10) Pub. No.: US 2011/0238163 A1
(43) Pub. Date: Sep. 29, 2011

(54) Multi-layered Device

(75) Inventors: GAVIN Paul ANDREWS, ANTRIM (IE); DAVID Simon JONES, Newtownabbey (IE); SEAN Patrick GORMAN, Downpatrick (IE)

(73) Assignee: LABORATORIOS FARMACEUTICOS ROVI S.A., Madrid (ES)

(21) Appl. No.: 13/043,021

(22) Filed: Mar. 8, 2011

Related U.S. Application Data
(63) Continuation-in-part of application No. PCT/GB09/51134, filed on Sep. 8, 2009.

(30) Foreign Application Priority Data
Sep. 8, 2008 (GB) GB0816365.1

(51) Int. Cl. A61L 27/58; C08F 220/14; C08B 15/00; C08G 77/04; A61L 33/04; A61L 29/14

(52) U.S. Cl. 623/1.46; 526/318.4; 536/56; 528/10; 427/2.25; 604/265; 604/500

(57) ABSTRACT

There is provided a device comprising a body structure having one or more surfaces wherein at least one of the surfaces comprises a pH sensitive layer comprising a linear polymer, wherein the water solubility of the linear polymer increases from a first water solubility to a second water solubility at a pH trigger. A method of forming a device, and a method of preventing or mitigating infection is also described.

### CROSS-REFERENCE TO EARLIER FILED APPLICATIONS

This application is a continuation-in-part of and claims the benefit of PCT International Application No. PCT/GB2009/051134 filed Sep. 8, 2009, which claims the benefit of British Application No. GB 0816365.1 filed Sep. 8, 2008, the entire disclosures of which are hereby incorporated by reference.

### FIELD OF THE INVENTION

The invention relates primarily to the field of medical devices. More specifically, the invention pertains to medical devices comprising pH sensitive degradable layers, methods of making medical devices containing pH sensitive layers and methods of using medical devices containing pH sensitive degradable, erodible or soluble layers. Multi-layered devices, such as a stent or catheter, comprising a structural layer and a pH sensitive layer are provided.

### BACKGROUND

The use of medical devices inserted into a patient's body is now routine in healthcare management within hospitals and nursing homes. Although there are substantial benefits associated with the use of inserted medical devices, such as, for example, catheters and stents, there are very worryingly a number of potentially dangerous complications that may lead to an increase in the time patients remain in hospital and more importantly in an increase in the number of patient deaths associated with the use of these devices. These complications arise principally because of the way in which a patient's body reacts to insertion of a medical device and what it perceives to be a foreign object. Consequently patients are often plagued by infection associated with the insertion of a medical device and this is seen to be one of the most critical disadvantages of an otherwise highly effective and beneficial medical treatment. There is an urgent need to improve what is often referred to as device-related infection.

Typically device-related infection begins with bacterial adherence (colonization, contamination), developing with the formation of biofilm. Bacteria and pathogens which typically colonize catheters produce urease which degrades urea in urine to form carbon dioxide and ammonia. At increased pH associated with such degradation/contamination, minerals in urine precipitate leading to encrustation.

Catheter encrustation can cause blockage of the catheter leading to an increase in the frequency with which the catheter must be removed and replaced. Encrustation also results in an increase in the pain of removal of the catheter. The tissue surrounding the catheter is also far more likely to become infected. This is particularly problematic for patients requiring long term catheterization. Serious consequences include septicemia, pyelonephitis and shock.

Additionally, associated pathogens within the biomass can compromise medical device lifetime through the expression of potent urease isoenzymes, which act to alkalinize urine through the conversion of urea to ammonia and carbon dioxide.

Previous approaches to overcoming this problem include the incorporation of antibiotics into the device to combat infection. After insertion, therapeutic agents may be released by diffusion and ultimately reside locally in the biological fluid adjacent to the device thus preventing bacterial adherence (contamination). Further, attempts have been made to modify device surfaces to reduce their susceptibility to infection.

Despite the attempts to alleviate the complications that plague the use of urinary devices, many problems still exist. Therefore, whilst antibiotic therapies and novel surface coatings may provide a temporary resolution, the only real definitive solution to the problems associated with urinary catheterization and ureteral stenting is device removal.

Medical devices, such as catheters, coated in lubricants are known. Lubricants comprising cross-linked hydrogels, including carboxylic acid functional groups are also known.

U.S. Pat. No. 6,306,422 discloses a device, particularly a urinary catheter, coated in a cross linked polymer hydrogel. At a trigger pH, the polymer swells through absorption of water. This absorption of water increases pore size of the hydrogel, enhancing the release of an active agent in a sustained release fashion. The polymer hydrogel of U.S. Pat. No. 6,306,422 remains water insoluble throughout, and remains coated onto the device. The active agent is typically one or more of an antibiotic and a urease inhibitor. The release of these active agents may control bacterial surface growth and control the formation of encrustation respectively. However, the release of a urease inhibitor does not remove any encrustation which has already formed on the catheter, because the cross-linked polymer is not water soluble or erodible under physiological conditions. In addition, the active agents released from devices disclosed in U.S. Pat. No. 6,306,422 would not be able to penetrate the biofilm formed by bacteria to remove existing bacterial colonization (contamination).

Coated catheters and stents have been disclosed: U.S. Pat. No. 5,607,417, U.S. Pat. No. 5,554,147, U.S. Pat. No. 5,788,687, U.S. Pat. No. 2,237,218, U.S. Pat. No. 3,566,874, U.S. Pat. No. 3,663,288, U.S. Pat. No. 3,695,921, U.S. Pat. No. 4,539,234, U.S. Pat. No. 4,557,724, U.S. Pat. No. 4,220,153, U.S. Pat. No. 4,026,296, U.S. Pat. No. 3,861,396, U.S. Pat. No. 3,580,983, U.S. Pat. No. 4,642,104, U.S. Pat. No. 4,773,901, U.S. Pat. No. 4,515,593, U.S. Pat. No. 4,392,848, U.S. Pat. No. 5,041,100, U.S. Pat. No. 5,360,415, U.S. Pat. No. 5,091,205, U.S. Pat. No. 4,906,237, U.S. Pat. No. 4,526,579, U.S. Pat. No. 5,464,650, U.S. Pat. No. 5,531,716, U.S. Pat. No. 5,591,227, U.S. Pub. 20010027340, U.S. Pub. 20020032414, U.S. Pub. 20020065546, U.S. Pub. 20020091375, U.S. Pub. 20020095133 and U.S. Pub. 20030040790.

### SUMMARY OF INVENTION

The inventors have developed a device surface that is inherently resistant to infection through the use of intelligent in vivo reactions and preferably impregnation with antibiotics. In particular, the device surface comprises shedding biomaterials (by erosion or dissolution in vivo or during use under physiological conditions in an aqueous environment), which can be shed in response to pH changes, as an alternative to currently utilized materials.

According to the first aspect of the present invention there is provided a device comprising a body structure having one or more surfaces comprising at least one pH sensitive degradable layer wherein the at least one pH sensitive degradable layer comprises a pH sensitive polymer wherein the pH sensitive degradable layer is capable of controlled degradation at a defined pH. In particular embodiments, the device can comprise a plurality of degradable layers. In some embodiments, the pH sensitive polymer comprises a linear pH sensitive polymer and excludes a cross-linked pH sensitive polymer.

According to the present invention, there is provided a device comprising a body structure having one or more surfaces wherein at least one of the surfaces comprises a pH sensitive layer comprising a linear polymer, wherein the water solubility of the linear polymer increases from a first water solubility to a second water solubility at a pH trigger.

Suitably, in embodiments, the device can be any device wherein the pH of fluid, for example bodily fluid, surrounding the device increases or decreases from a defined value, for example wherein pH can increases or decreases from physiological pH in response to infection.

At the pH trigger, the linear polymer ionizes, and this causes the water solubility of the polymer to increase. The ionized linear polymer then dissolves into the aqueous environment surrounding the device, and a new surface of the device is revealed. The new surface does not have any bacteria colonized thereon, and will be free of encrustation. The device of the present invention can thus remain implanted for extended periods of time compared to prior art devices. The risk of infection and encrustation is also reduced as the surface of the device of the present invention is shed once colonized with bacteria to any significant extent, and a new surface is exposed.

The device of the present invention may typically be a urinary catheter or urinary stent. Bacteria which typically colonizes such devices release urease. Urine degrades into ammonia and carbon dioxide upon contact with urease, substantially increasing the pH of the area surrounding the catheter. This increase in pH causes minerals to precipitate from urine, causing encrustation of the catheter. The increase in pH generated through the production of ammonia triggers the linear polymer of the present invention to ionise, and the water solubility of the polymer increases accordingly. The linear polymer dissolves into the surrounding aqueous environment, and any encrustation present on the outer surface of the device is removed with the linear polymer. A new surface of the device is exposed. The new surface is free of bacteria and encrustation.

Alternatively, the device of the present invention may be in the form of dental braces or dentures. Upon bacterial colonization of such devices, the surrounding pH decreases to below physiological pH. As bacteria in the oral cavity produce acid, the greater the degree of bacterial colonization, the greater the amount of acid produced, lowering the surrounding pH.

The linear polymer of the present invention can be capable of changing from providing a stable layer to a layer which undergoes controlled erosion or dissolution as the pH of the surrounding environment moves away from physiological pH, wherein physiological pH is typically 6.2. Suitably, such erosion or dissolution occurs towards the endpoints of the range pH 5.5 to pH 7. In embodiments, a pH sensitive polymer can be capable of controlled dissolution, degradation or erosion at a pH indicative of infection, being removed from physiological pH, for example a pH greater than pH 6.2, such as pH 6.5 or pH 7 or higher. In other embodiments, the pH indicative of bacterial infection is less than pH 6.2, such as pH 6 or pH 5.5 or lower.

The pH trigger depends on the intended environment surrounding the device of the present invention. Where the device is intended to be implanted into a neutral or alkali environment such as the urinary bladder, the pH trigger is typically above 6.5; suitably above 7; more suitably approximately 7.2. Where the device is intended to be implanted into an acidic environment such as the stomach, the pH trigger is typically less than 6.0; suitably less than 5.5.

Where the pH of the area surrounding the device of the present invention moves away from the trigger pH and towards physiological pH, the water solubility of the pH sensitive layer may decrease accordingly, and move towards the first water solubility. As such, the rate of dissolution or erosion of the pH sensitive layer may decrease. According to one embodiment, the pH sensitive layer dissolves or erodes only where the device is colonized by bacteria. The dissolution or erosion of the pH sensitive layer may start and stop depending on the colonization of the device. In other words, a “pH trigger” or “trigger pH” is a pH value of the environment surrounding or immediately adjacent the device which pH is indicative of bacterial contamination and which pH causes an increase in the solubility of the pH sensitive polymer (layer).

Suitably, in embodiments, such a pH sensitive layer can provide for a first rate of release of functional excipients and/or active agents at physiological pH (for example pH 6.2) and at a second rate at non physiological pH (for example pH 7.0). Generally the first rate is lower than the second rate. In embodiments where shedding of the pH sensitive layer is minimal at physiological pH and increased at a pH removed from physiological pH, elution of the functional excipients and/or active agents may be correlated with erosion or dissolution of the pH sensitive layer. This can be advantageous as infection can move pH away from physiological values and thus the release of the functional excipients and/or active agents may correlate with infection.

Suitably, in preferred embodiments the linear polymer is biocompatible. A “biocompatible” material is a material that is compatible with living tissue or a living system by not being toxic or injurious. In particular embodiments the device may comprise material which forms biostable layers or “structural layers”. A “non-bioabsorbable” or “biostable” material refers to a material, such as a polymer or copolymer, which remains in the body without substantial bioabsorption. Such structural layers may be included in the device of the present invention to provide structural support to the device and to provide a body (surface) upon which one or more layers of linear pH sensitive polymer can be applied or built. A structural layer generally comprises one or more biocompatible and biostable materials.

A structural layer can comprise one or more of the following materials: silicone, latex, (poly (vinyl chloride)), polyurethane, ethylene-vinylacetate copolymer, polyethylene, polypropylene, polyester, polystyrene, nylon.

Suitably, in embodiments, the linear polymer undergoes structural changes with respect to changes in pH, in particular the linear polymer may become ionized with changes in pH, causing the linear polymer to have increased water solubility.

The polymers for use in the device of the present invention are generally linear rather than cross-linked. The linear polymer of the present invention becomes ionized at a pH trigger, causing the water solubility of the linear polymer to greatly increase. In contrast, cross-linked polymers ionize at a pH trigger causing the polymers to absorb water and swell. The water solubility of the cross-linked polymer is not altered through ionization, and even following ionization the cross-linked polymer is retained on devices such as those disclosed in U.S. Pat. No. 6,306,422. Such cross-linked polymers are generally in the form of hydrogels. The linear polymers of the device of the present invention are generally extrudable.

Typically, the pH sensitive layer absorbs less than 30 wt % water prior to ionization; generally less than 20 wt %, suitably less than 10 wt %. In contrast, prior art cross-linked hydrogels for use in connection with devices such as those disclosed in U.S. Pat. No. 6,306,422 absorb up to several thousand times their weight in water prior to ionization. The water solubility of the hydrogels is not affected, and these hydrogels maintain their shape and do not dissolve into the surrounding aqueous environment. Although the polymers for use in the device of the present invention may swell with absorption of water prior to ionization, this precedes ionization resulting in an increase in water solubility and dissolution into the surrounding aqueous environment.

Where structural changes in the linear polymer are intended to be triggered at a pH of at least 6.5, the linear polymer typically comprises one or more carboxylic groups. The linear polymer may be a pH sensitive cellulose polymer comprising an ionizable cellulose polymer derivative, for example a derivatized cellulose ester or derivatized cellulose ether, wherein the cellulose ester or ether is selected from the group consisting of hydroxylethylcellulose (HEC), methylcellulose (MC), hydroxypropylcellulose (HPC), cellulose acetate (CA), cellulose acetate butyrate (CA), and hydroxypropyl methylcellulose (HPMC), and the cellulose ester or ether is derivatized with one or carboxylic acid functional groups or with one or more functional groups comprising one or more carboxylic acid moieties.

According to some embodiments, the polymer is a linear cellulose derivative having the following structure:

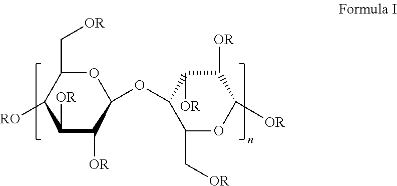

wherein:
n is 2 to about 10,000, about 150 to about 10,000, about 250 to about 1500, about 3500 to about 7,500, about 150 to about 850, about 1,000 to about 3,000, about 6,500 to about 7,000, about 8,000 to about 10,000 or about 400 to about 5,000;
at least one R is selected from the group consisting of —H, —CH₃, —COCH₃, —CH₂CH(OH)CH₃, —COCH₂CH₂CH₃ and —CH₂CH(OCOCH₃)CH₃; and
at least one R is selected from the group consisting of —CH₂CH₂CH₂COOH, —CH₂CH(OCOCH₂CH₂COOH)CH₃, —COCH₂CH₂COOH, —CO(C₆H₄)CO₂H, —CH₂CH(OCO(C₆H₄)CO₂H)CH₃ and —CO(C₆H₃)(CO₂H)₂.

Some embodiments of the pH sensitive linear polymer include those wherein: a) at least one R is H, at least one R is —CH₃, at least one R is —CH₂CH(OH)CH₃, at least one R is —COCH₃, and at least one R is —COCH₂CH₂COOH, optionally wherein at least one R is —CH₂CH(OCOCH₂CH₂COOH)CH₃ or at least one R is —CH₂CH(OCOCH₃)CH₃ (HPMC-AS); b) at least one R is H, at least one R is —CH₃, at least one R is —CH₂CH(OH)CH₃, and at least one R is —CO(C₆H₄)CO₂H, optionally wherein at least one R is —CH₂CH(OCO(C₆H₄)CO₂H)CH₃ (HPMCP); c) at least one R is H, at least one R is —COCH₃, and at least one R is —CO(C₆H₄)CO₂H(CAP); d) at least one R is H, at least one R is —COCH₃, and at least one R is —COCH₂CH₂CH₃ (CAB); e) at least one R is H, at least one R is —COCH₃, and at least one R is —CO(C₆H₃)(CO₂H)₂ (CAT).

In some embodiments the linear pH sensitive polymer is an ionizable linear cellulose derivative comprising one or more carboxylic acid functional groups, the polymer being selected from the group consisting of hydroxypropyl methylcellulose acetate succinate (HPMC-AS, otherwise known as hypromellose acetate succinate), hydroxypropyl methylcellulose phthalate (HPMCP, otherwise known as hypromellose phthalate), cellulose acetate trimellitate (CAT), cellulose acetate phthalate (CAP), cellulose acetate butyrate (CAB).

HPMCP is a phthalate ester of HPMC and contains not less than 21% wt. and not more than 35% wt. phthalyl groups. Suitable grades of HPMCP include: a) dissolution above pH 5 with a phthalate content of about 24% wt; b) dissolution above pH 5.5 with a phthalate content of 31%). Specific grades are detailed in the table below, wherein the pH value required for dissolution (the trigger pH) is as specified, which grades are supplied by Shin-Etsu Chemical Company (Tokyo, Japan).

| | Nominal Phthalyl | Labeled Viscosity | |
|---|---|---|---|
| Grade | Content (% wt.) | (cSt)* | Trigger PH |
| HPMCP 50 | 24 | 55 | ≧5 |
| HPMCP 55 | 31 | 40 | ≧5.5 |
| HPMCP 55S | 31 | 170 | ≧5.5 |

*Determined at 10% wt. in a mixture of equal weights of ethanol and methylene chloride according to USP/NF measuring method.

The above grades of HPMCP are further described as detailed in the table below.

| | Nominal | Nominal | Nominal | |
|---|---|---|---|---|
| | Phthalyl | Hydroxypropoxyl | Methoxyl | Trigger |
| Grade | Content (%) | Content (%) | Content (%) | pH |
| HPMC-P | 27-35 | 5-9 | 18-22 | >5 |
| HP-55 | | | | |
| HPMC-P | 27-35 | 5-9 | 18-22 | ≧5.5 |
| HP-55S | | | | |
| HPMC-P | 21-27 | 6-10 | 20-24 | ≧5.5 |
| HP-50 | | | | |

HPMC-AS contains not less than 12% wt. and not more than 28% wt. of methoxy groups (—OCH₃), not less than 4% wt. and not more than 23% wt. hydroxypropoxy groups (—OCH₂CH(OH)CH₃), not less than 2% wt. and not more than 16% wt. acetyl groups (—COCH₃), and not more less 4% wt. and not more than 28% wt. succinoyl groups (—COCH₂CH₂COOH). Suitable grades of HPMC-AS include: a) dissolution above 5.5, acetyl content 8%, succinoyl content 15%; b) dissolution above 6.0, acetyl content 9%, succinoyl content 11%; c) dissolution above 6.8, acetyl content 12%, succinoyl content 7%. Specific suitable grades are detailed in the table below, wherein the pH value required for dissolution (the trigger pH) is as specified. The HPMC-AS grades LF/MF/HF, having an approximate molecular weight 18000 g/mol, are supplied by Shin-Etsu® Chemical Co. (Tokyo, Japan) under the brand name AQOAT®.

| | Acetyl Content | Succinyl Content | Trigger |
|---|---|---|---|
| Micronized Grade | (% wt.) | (% wt.) | PH |
| HPMC-AS LF (or LG) | 8 | 15 | ≧5.5 |
| HPMC-AS MF (or MG) | 9 | 11 | ≧6 |
| HPMC-AS HF (or HG) | 12 | 7 | ≧6.8 |

Other suitable grades of HPMC-AS supplied by Shin-Etsu are detailed in the table below:

| | Nominal | Nominal | Nominal | Nominal | |
|---|---|---|---|---|---|
| | Succinoyl | Acetyl | Hydroxy- | Methoxyl | |
| | Content | Content | propoxyl | Content | Trigger |
| Grade | (%) | (%) | Content (%) | (%) | pH |
| HPMC-AS | 14-18 | 5-9 | 5-9 | 20-24 | >5 |
| AS-L | | | | | |
| HPMC-AS | 10-14 | 7-11 | 5-9 | 21-25 | ≧5.5 |
| AS-M | | | | | |
| HPMC-AS | 4.0-8.0 | 10-14 | 6-10 | 22-26 | ≧5.5 |
| AS-H | | | | | |

Cellulose acetate phthalate is commercially available from Eastman Chemical (Kingsport, Tenn.) in the following grade.

| | Nominal | Nominal | Nominal | |
|---|---|---|---|---|
| | Phthalyl | Hydroxyl | Acetyl | Trigger |
| Grade | Content (%) | Content (%) | Content (%) | pH |
| CAP | 35 | 0-1 | 24 | >6.2 |

Cellulose acetate butyrate is commercially available from Eastman Chemical (Kingsport, Tenn.) in the following grade.

| | Nominal | Nominal | Nominal | |
|---|---|---|---|---|
| | Butyryl | Hydroxyl | Acetyl | Trigger |
| Grade | Content (%) | Content (%) | Content (%) | pH |
| CAB | 37 | 1.5 | 13 | >6.0 |

Cellulose acetate trimellitate is commercially available from Eastman Chemical (Kingsport, Tenn.) in the following grade.

| | Nominal | Nominal | Nominal | |
|---|---|---|---|---|
| | Trimellityl | Hydroxyl | Acetyl | Trigger |
| Grade | Content (%) | Content (%) | Content (%) | pH |
| CAB | 37 | 1.5 | 13 | >5.0 |

In one embodiment, the linear polymer may be a methacrylate polymer or a polymer or copolymer comprising methacrylate. In embodiments of the invention the linear polymer can be selected from acrylate polymers, acrylate copolymers, methacrylate polymers, methacrylate copolymers, and derivatives thereof. In embodiments, the linear polymer can be selected from the group comprising, for example, Eudragit® L100 (dissolution above pH 6.0; anionic copolymer based upon methacrylic acid and methyl methacrylate; poly(methacrylic acid-co-methyl methacrylate) 1:1), Eudragit® 5100 (dissolution above pH 7.0; anionic copolymer based upon methacrylic acid and methyl methacrylate; poly(methacrylic acid-co-methyl methacrylate) 1:2). Eudragit® polymers and copolymers are available from Evonik® Degussa Corporation (Parsippany, N.J.).

In some embodiments, the linear polymer excludes a cross-linked methacrylate polymer, cross-linked methacrylate copolymer, cross-linked acrylate-co-methacrylate copolymer and/or cross-linked acrylate copolymer. As used herein and unless otherwise specified, the term methacrylate refers to methacrylic acid, methacrylic acid salt, or methacrylate ester and their derivatives. As used herein and unless otherwise specified, the term acrylate refers to acrylic acid, acrylic acid salt, or acrylate ester and their derivatives. Exemplary derivatives are described herein.

In embodiments, the linear polymer can be an Eudragit® polymer. Eudragit® polymers can be provided as a single system or blends of two different types. In embodiments Eudragit® L100, 5100 and combinations of these polymers can be used.

In embodiments, Eudragit® L100 can be used to provide a layer which is capable of erosion at pH values greater than 6.

In embodiments, Eudragit® S100 can be used to provide a layer which erodes at pH values exceeding (or) 7.0.

As will be appreciated, pH sensitive layers of a device can be manufactured using a combination of both L100 and 5100 to generate systems that erode slowly under normal urinary conditions, but will rapidly shed at higher pH values. Layers of different pH sensitive polymers for example layers of Eudragit® L100, 5100 and combinations of these polymers can be used to form a device such that different layers in a device erode at different pH levels.

Where structural changes in the linear polymer are intended to be triggered at a pH of less than 6.0, the linear polymer typically comprises primary, secondary and tertiary amines, typically —NH₂ groups; suitably the polymer comprises diethylaminoacrylate, dimethylaminoethylacrylate and/or other acrylate monomers. Typically the polymer is a copolymer of dimethylaminomethacrylate and other acrylate monomers. Suitably the polymer is that sold under the trade mark Eudragit® E100 (soluble in gastric fluid up to pH 5.5; cationic copolymer based upon dimethylaminoethyl methacrylate, butyl methacrylate and methyl methacrylate; poly(butyl methacrylate-co-(2-dimethylaminoethyl methacrylate-co-methyl methacrylate) 1:2:1).

As noted above, the water solubility of the linear polymer of the device of the present invention increases from a first water solubility to a second water solubility at a pH trigger.

Typically at a pH of the pKa of the polymer, 50% of the polymer or more is ionized.

According to one aspect of the present invention the second water solubility of the linear polymer is at least 200% more than the first water solubility of the linear polymer, generally at least 400% more, typically at least 600% more.

Advantageously, upon dissolution or erosion the polymer chain of the linear polymer remains intact, comprising the same monomer units as before dissolution or erosion.

The device of the present invention may be any device wherein a change in pH is associated with colonisation of the device with bacteria. In embodiments of the present invention, the device is a medical device, for example an intracorporeal or extracorporeal device including catheters, temporary or permanent implants, stents, grafts, repair devices, and implantable devices.

Typically the device is a catheter, suitably a urinary catheter, a urethral stent, a naso-gastric tube, a CAPD tube (continuous ambulatory peritoneal dialysis catheter), a bilary stent, dental braces or dentures.

Advantageously, the device of the present invention is a urinary catheter or a urethral stent.

The structural layer and pH sensitive layer (otherwise termed “erodible layer”) of the embodiments described herein can further comprise one or more functional excipients. In some embodiments, one of the layers of the device comprises one or more functional excipients. In some embodiments, two or more layers of the device comprise one or more functional excipients. In some embodiments, all of the layers of the device comprise one or more functional excipients. In some embodiments, all of the layers of the device exclude one or more functional excipients.

The pH sensitive layer may comprise one or more functional excipients, one or more active agents or a combination thereof to be released with the dissolution or erosion of the pH sensitive layer. The functional excipients may suitably be buffer groups (organic acids) such as citric acid, tartaric acid, succinic acid, and fumaric acid, EDTA and plasticizing agents, for example triethyl citrate, and tributyl citrate, other standard pharmaceutical excipients used to facilitate manufacture or performance, or combinations thereof. Exemplary active agents can be selected from the group consisting of antimicrobial compounds, Levofloxacin and Nalidixic acid, antibiotic compounds, chlorhexidine, povidone-iodine, tridosan, urease inhibitors, or combinations thereof.

In some embodiments, the functional excipient can be present in a layer of the device in an amount ranging from 0.5-50% wt. or 5-35% wt. of the layer.

In some embodiments, the active agent can be present in a layer of the device in an amount ranging from 0.1-40% % wt. or 5-15% wt. of the layer.

Typically the functional excipients and/or active agents are released in a sustained release manner following implantation of the device. According to one embodiment, the rate of release of the functional excipients and/or active agents may increase sharply upon erosion or dissolution of the pH sensitive layer.

The functional excipient and/or active agent may be adsorbed directly to the linear polymer, or may be disposed inside the device or otherwise associated with it via the use of one or more linker molecules or other attachment means including covalent, ionic, van der Waals bonds. The pH sensitive layer and/or surface may be configured such that controlled release of the functional excipient and/or active agent occurs, for example the functional excipient and/or active agent elutes slowly over time. By “controlled release” is meant an alteration of the rate of release of a therapeutic agent or functional excipient and/or active agent from a medical device coating in a given environment. This may be accomplished using time released coatings, for example. In embodiments of the device of the invention, layers are provided which are adapted to simultaneously release therapeutic agent(s) at two or more different rates from different portions of a layer or at two different rates depending on the pH surrounding the device.

In embodiments, the device can include layers, which can include pH sensitive polymer layers, which are loaded with a functional excipient and/or active agent, in particular a drug, for example an antibiotic. Alternatively, in embodiments the medical device or the medical device coating comprising the pH sensitive layer degrades in a controlled manner relative to pH and the drug can be bound to the linear polymer. By incorporating a drug within the material of the medical device or the material coating the medical device, the drug is dispensed in a gradual manner as the layer comprising the pH sensitive polymer degrades.

Suitably, in embodiments the dissolution of a pH sensitive layer triggers the release of an antibiotic.

In embodiments, the device of the present invention can comprise a drug which minimizes bacterial adhesion to the device or growth of a pathogen on the device, for example an antibiotic. An advantage of the device of the present invention is that it allows much higher drug concentrations at the site of infection in comparison to conventional routes of drug therapy such as orally swallowed tablets.

The release of an antibiotic may control bacterial growth on the surface of the device. However, a biofilm is generally formed once bacteria have effected colonization of a device. Antibiotic compounds cannot generally penetrate such biofilms, and are therefore not very effective at removing such bacterial biofilms. The release of a urease inhibitor acts to control the growth of encrustation but does not remove encrustation which has already formed. The surface of the device of the present invention starts to dissolve or erode at a trigger pH, and bacterial colonization (contamination) and encrustation is removed with the surface. A new surface is revealed free of all bacterial colonization and encrustation.

In effect a device of the present invention can self-cleanse once infected with bacteria, that is to say should drug elution fail to inhibit bacterial adherence and subsequently result in increased urinary pH by the action of urease on urea, a layer of the device is capable of recognizing the formation of microbial biofilm and initiate controlled erosion (regulated by the incorporation of organic acids, such as citric acid) and thus remove any adherent masses. In so doing, the device surface will be cleansed and the functional agents (EDTA & citric acid) and/or active agent(s) incorporated into the film will be released to the device/fluid interface. This will regulate the urinary pH by the action of citric acid and very importantly sequester Ca²⁺ and Mg²⁺ metal ions. This process will renew the device surface, return the pH to normal values and ‘mop up’ metal ions that are pertinent to the formation of crystalline deposits on the device surface.

This enables the devices and ultimately the patients to remain free from infection for the duration the device is to be used.

Upon release of the functional excipients and/or active agent(s), the bacterial colonization of the device may be reduced, and the pH of the area surrounding the device may move away from the trigger pH and towards physiological pH. The water solubility of the pH sensitive layer may decrease towards the first water solubility accordingly.

The linear polymer absorbs water at a trigger pH, causing ionization. The rate of ionization may be engineered by controlling the rate of absorption of water, typically by controlling the density of the pH sensitive polymer layer. A decreased density of linear polymer in the pH sensitive layer leads to a decreased rate of ionization.

The rate of ionization depends on the composition of the pH sensitive layer, as well as the pH of the area surrounding the device.

According to one embodiment the pH sensitive layer comprises a second or third hydrophilic polymer. Suitable hydrophilic polymers include polyethylene oxide, polyacrylic acids and/or cellulose derivatives (particularly linear cellulose derivatives) such as hydroxypropylcellulose, hydroxypropyl methylcellulose and polyvinylpyrrolidone. The addition of one or more hydrophilic polymers to the pH sensitive layer provides physical interactions such as Van der Waals interactions. As used herein a derivative is a chemical substance related structurally to another substance with which it is named, i.e. a parent substance, and is theoretically derivable from it. A derivative is also a compound that is obtained by chemical modification of a parent compound such that the “derivative” includes within it almost all or all of the chemical structure of the parent (or base) compound. A derivative is also a compound derived or obtained from a parent compound and containing essential elements of the parent compound.

Alternatively, the pH sensitive layer may comprise a hydrophobic polymer, in particular a low molecular weight hydrophobic polymer. Suitable hydrophobic polymers include polylactic acid, polyglycolic acid, polylactide-co-glycolide and polycaprolactone. Generally the hydrophobic polymer is substantially homogenously dispersed in the pH sensitive layer.

The rate of dissolution or erosion of the pH sensitive layer is dependent on its composition. Buffer groups may be incorporated into the pH sensitive layer to reduce the rate of dissolution or erosion. Suitable buffer groups include citric acid, tartaric acid, succinic acid, fumaric acid and related compounds. Where the extent of bacterial colonization is low, the number of ions released is low. Some of these ions will be taken up by the buffer group resulting in a lower rate of degradation, and increasing the lifetime of the device of the present invention. The number of ions released will increase with increased bacterial colonization leading to the erosion or dissolution of the pH sensitive layer regardless of the incorporation of the buffer group.

According to one aspect, the device of the present invention may comprise more than one pH sensitive layer; typically more than three pH sensitive layers; more suitably five pH sensitive layers.

Each pH sensitive layer may have the same pH trigger or a different pH trigger.

Each pH sensitive layer may have a different second water solubility. Alternatively each pH sensitive layer may have the same second water solubility.

Each pH sensitive layer may have the same rate of ionization and the same rate of dissolution or erosion. Alternatively, different pH sensitive layers may have the same or different rates of ionization and/or rates of dissolution or erosion.

According to one embodiment, adjacent pH sensitive layers may have the same pH trigger, but different second water solubilities.

Alternatively, adjacent pH sensitive layers may have different pH triggers, but the same second water solubilities.

Typically adjacent pH layers have different rates of ionization, or different rates of dissolution or erosion.

According to one embodiment, different pH sensitive layers comprise different functional excipients and/or active agent(s) to be released upon dissolution or erosion of the pH sensitive layer.

According to one embodiment, the device may comprise a lubricating layer to increase its ease of insertion and removal. Typically the lubricating layer may comprise one or more cross-linked polymers.

Generally the device comprises an inside surface and an outside surface, said inside surface defining a lumen.

Typically the outside surface of the device comprises the lubricating layer. Generally the inside surface of the device comprises the pH sensitive layer.

Generally the device comprises at least one structural layer which is substantially non-degradable or erodable in the body and provides structural stability to the device regardless of the pH of the surrounding environment.

Typically the water solubility of the structural layer remains substantially constant between a pH of 2 to 10. Generally the water solubility of the structural layer remains substantially constant regardless of the pH of the surrounding environment.

In particular embodiments the device can comprise a two layer system wherein the pH sensitive layer, for example an Eudragit® layer is provided on the inside of a two layer system. In such a system, a fluid, for example a bodily fluid such as urine, can flow through the inner lumen of the device (FIG. 2a).

In alternative embodiments the device can comprise a three layer system in which two pH sensitive layers, for example Eudragit® layers, form the inner and outer layers of the device. In such three layer systems, a fluid, for example a bodily fluid such as urine, can flow through the inner lumen and over the outer surface of the device (FIG. 2b).

In further alternative embodiments, devices comprising a plurality of pH sensitive layers can be provided. In particular embodiments a first pH sensitive layer can be provided adjacent to a second pH sensitive layer such that on erosion of the first layer, the second layer is exposed. In embodiments, the pH sensitive layers can be capable of erosion at different pH values.

Suitably, in embodiments the inner and outer layers of the device can be melt extruded.

Suitably, in embodiments a degradable layer can be amenable to insertion and removal of the device from within the body of a patient. In embodiments, where necessary, a structural layer comprising suitable polymers can be provided in combination with a degradable layer in a device.

According to a further aspect of the present invention there is provided a coating for application to a device, the device comprising a body structure having one or more surfaces and the coating being adapted to be applied to at least one surface of the device such that when a surface of the device is provided with at least one coating, a pH sensitive layer is provided on the device, said pH sensitive layer comprising a linear polymer wherein the water solubility of the linear polymer increases from a first water solubility to a second water solubility at a pH trigger.

In particular embodiments the device can comprise a coating comprising a plurality of pH sensitive layers.

The term “coating,” as used herein and unless otherwise indicated, refers generally to material attached to a device. A coating can include material covering any portion of a medical device, and can be configured as one or more coating layers. A coating can have a substantially constant or a varied thickness and composition. Coatings can be adhered to any portion of a device surface, for example a medical device including the luminal surface, the abluminal surface, or any portions or combinations thereof.

Generally by pH sensitive layer is meant a layer which can be dissolved or eroded at a defined pH, for example wherein the polymer can be ionized at higher pH levels such that the water solubility of the polymer increases. Suitably, after sufficient dissolution or erosion, a complete layer may be removed such that a new layer in the device is exposed. Generally upon dissolution or erosion of the pH sensitive layer, the polymer chain remains intact with the same monomer units.

In embodiments, the pH sensitive layer includes functional excipients such as citric acid or other small organic molecules and such functional excipients will be released upon dissolution or erosion of the pH sensitive layer, generally in a controlled release fashion.

An aspect of the invention provides a multilayered catheter or stent comprising plural coextensive and centrically arranged layers, said layers defining a lumen, wherein:
a first coextensive layer is a structural layer that is substantially non-degradable or non-erodible under physiological conditions; and
at least one second coextensive layer is a pH sensitive layer comprising one or more pH sensitive linear polymers that ionize and dissolve, erode or degrade in an aqueous environment when the second coextensive layer is contaminated with bacteria.

Some embodiments of the invention includes those wherein: a) the at least one second coextensive layer is in the interior of the first coextensive layer; b) the at least one second coextensive layer is at the exterior of the first coextensive layer; c) at least one second coextensive layer is in the interior of the first coextensive layer, and at least one second coextensive layer is at the exterior of the first coextensive layer; d) two second coextensive layers are in the interior of the first coextensive layer, and one second coextensive layer is at the exterior of the first coextensive layer; e) two interior second coextensive layers ionize and dissolve, erode or degrade at different pH values in an aqueous environment; f) all second coextensive layers ionize and dissolve, erode or degrade at the same pH values in an aqueous environment; g) two second coextensive layers are at the exterior of the first coextensive layer, and one second coextensive layer is in the interior of the first coextensive layer; h) two exterior second coextensive layers ionize and dissolve, erode or degrade at different pH values in an aqueous environment; i) two second coextensive layers are in the interior of the first coextensive layer, and two second coextensive layers are at the exterior of the first coextensive layer; j) two interior second coextensive layers ionize and dissolve, erode or degrade at the same pH values in an aqueous environment; and/or k) two exterior second coextensive layers ionize and dissolve, erode or degrade at the same pH values in an aqueous environment.

According to a further aspect of the present invention there is provided a method of forming a device comprising the steps of providing a structural layer and applying at least one pH sensitive layer thereto, said pH sensitive layer comprising a linear polymer wherein the water solubility of the linear polymer increases from a first water solubility to a second water solubility at a pH trigger.

Typically the structural layer has an inside surface and an outside surface, said inside surface defining a lumen. Generally the pH sensitive layer is applied to the inside surface of the structural layer. The method may comprise the step of applying more than one pH sensitive layer.

Generally the device is a medical device, suitable for insertion or implantation into the human or animal body.

In particular embodiments of the second aspect of the invention, the method includes multi-layer extrusion. A multi-layered device of the invention can be prepared by multi-layer extrusion. Accordingly, a pH sensitive layer can be extruded onto or within a structural layer or onto or within another pH sensitive layer.

Suitably the device as described above is formed according to the method of the present invention.

According to a further aspect of the present invention there is provided a method of preventing or mitigating infection associated with a device implanted or inserted into the human or animal body comprising the step of implanting or inserting a device into the human or animal body, said device comprising a pH sensitive layer comprising a linear polymer, wherein the water solubility of the linear polymer increases from a first water solubility to a second water solubility at a pH trigger.

Generally the method results in any infection already formed being removed.

Generally the method includes the step of preventing or mitigating the formation of encrustation of the device. Typically the method also includes the step of the removal of any encrustation already formed.

Typically the time for which the device is implanted or inserted into the human or animal body without associated infection is at least 1 day, generally at least 3 days, suitably 7 days or more.

According to one embodiment, the time of implantation or insertion may be increased by at least 100% compared to equivalent devices which do not comprise at least one pH sensitive layer. Generally the time of insertion or implantation may be increased by at least 150%; typically at least 200%.

Typically the method of the present invention prevents or mitigates infection associated with the insertion or implantation of a catheter, in particular a urinary catheter, a stent, in particular a urethral stent or a bilary stent, an implantable or insertable tube, in particular a naso-gastric tube or a CAPD tube, dental braces or dentures.

According to a further aspect of the present invention there is provided a method of preventing or mitigating infection associated with a device implanted or inserted into the human or animal body comprising the steps of applying at least one pH sensitive layer to the device, said pH sensitive layer comprising a linear polymer wherein the water solubility of the linear polymer increases from a first water solubility to a second water solubility at a pH trigger.

According to a further aspect of the present invention there is provided a device for use in therapy, said device comprising at least one pH sensitive layer comprising a linear polymer wherein the water solubility of the linear polymer increases from a first water solubility to a second water solubility at a pH trigger.

Generally the therapy is preventing or mitigating infection associated with the insertion or implantation of the device in a human or animal body.

According to a further aspect of the present invention, there is provided a device for use in therapy, said device comprising a pH sensitive layer comprising a linear polymer, wherein the water solubility of the linear polymer increases from a first water solubility to a second water solubility at a pH trigger.

According to a further aspect of the present invention, there is provided the use of a device for the prevention or mitigation of infection, said device comprising at least one pH sensitive layer comprising a linear polymer wherein the water solubility of the linear polymer increases from a first water solubility to a second water solubility at a pH trigger.

According to a further aspect of the present invention, there is provided the use of a device in the manufacture of a medicament for the prevention or mitigation of infection, said device comprising at least one pH sensitive layer comprising a linear polymer wherein the water solubility of the linear polymer increases from a first water solubility to a second water solubility at a pH trigger.

According to a further aspect of the invention there is provided a pH sensitive polymer wherein said polymer shows a variable drug elution profile in the pH range pH 5 to pH 7.8, more preferably in the pH range pH 6 to pH 7.2 or alternatively in the pH range 5 to 6, preferably 5.5 to 6. The pH sensitive polymer is generally linear.

By a variable drug elution profile is meant that drug is eluted from a polymer at a first rate at a first end of a given pH range and a second different rate at a second opposite end of a given pH range. As a pH sensitive polymer undergoes degradation, for example becomes more soluble at a given pH, for example a pH removed from physiological pH, drug release can occur. The greater the degradation, for example at more extreme pH values removed from physiological pH, the greater the release of drug.

According to a further aspect of the invention there is provided a pH sensitive polymer wherein said polymer has a change in structural integrity in the pH range pH 5 to pH 7.8, more preferably in the pH range pH 6 to pH 7.2 or alternatively in the pH range 5 to 6, preferably 5.5 to 6. The pH sensitive polymer is generally linear.

By change in structural integrity it is meant the polymer is able to form sheets or layers of polymer about one end of the pH range, but is degraded and unable to form sheets or layers of polymer at an opposite end of the pH range.

According to a further aspect of the present invention, there is provided methods of use for treating patients with any one or more of the medical devices disclosed herein, which include, for example, a method of therapeutically treating a patient comprising contacting the patient with a medical device comprising a body structure having one or more surfaces comprising at least one pH sensitive layer wherein the at least one pH sensitive layer comprises a linear polymer wherein the water solubility of the linear polymer increases from a first water solubility to a second water solubility at a pH trigger. Methods are disclosed for administering a drug compound to a body of a patient which comprises, for example, providing a drug-eluting device of the present invention.

In another related embodiment of the invention, a method of administering a composition to a patient is disclosed which comprises providing a composition-eluting device, and introducing the composition-eluting device into the body of the patient, wherein the composition-eluting device comprises a body structure having one or more surfaces comprising at least one pH sensitive layer wherein the at least one pH sensitive layer comprises a linear polymer wherein the water solubility of the linear polymer increases from a first water solubility to a second water solubility at a pH trigger.

An aspect of the invention provides a device comprising an extended body structure, such as a tube, comprising:
an extended structural layer having an inside surface and an outside surface, said inside surface defining an extended lumen, wherein the structural layer is substantially non-degradable or erodable at a pH of 2 to 10 and provides structural stability to the device regardless of the pH of a surrounding aqueous environment of use;
at least one extended pH sensitive degradable layer attached to, in centric arrangement with, and coextensive with the structural layer and being capable of controlled erosion, dissolution or degradation at a pH in the range of 5.5 to 7, each degradable layer comprising a pH sensitive linear polymer that ionizes and dissolves in an aqueous environment of use when the pH of the environment moves away from an initial pH, e.g. pH 6.2, to a pH that is indicative of infection, e.g. bacterial contamination, wherein the water solubility of the linear polymer increases from a first water solubility to a second water solubility at a pH trigger.

Some embodiments of the invention include those wherein: a) the at least one extended pH sensitive degradable layer is disposed in the interior of the structural layer; b) the at least one extended pH sensitive degradable layer is disposed on the exterior of the structural layer; c) plural pH sensitive degradable layers are present; d) at least one extended pH sensitive degradable layer is disposed on the exterior of the structural layer and at least one extended pH sensitive degradable layer is disposed in the interior of the structural layer; e) plural extended pH sensitive degradable layers are disposed on the exterior of the structural layer and plural extended pH sensitive degradable layers are disposed in the interior of the structural layer; f) plural extended pH sensitive degradable layers are disposed on the exterior of the structural layer and at least one extended pH sensitive degradable layer is disposed in the interior of the structural layer; g) at least one extended pH sensitive degradable layer is disposed on the exterior of the structural layer and plural extended pH sensitive degradable layers are disposed in the interior of the structural layer.

Some embodiments of the invention include those wherein: a) plural extended pH sensitive degradable layers are present and at least two of those layers comprise different pH sensitive linear polymers; b) plural extended pH sensitive degradable layers are present and at least two of those layers erode, dissolve or degrade at different pH triggers during use under physiological conditions; c) at least one extended pH sensitive degradable layer is disposed on the exterior of the structural layer, at least one extended pH sensitive degradable layer is disposed in the interior of the structural layer and the layers erode, dissolve or degrade at different pH triggers during use under physiological conditions; d) plural extended pH sensitive degradable layers are disposed on the exterior of the structural layer and at least two of the layers erode, dissolve or degrade at different pH triggers during use under physiological conditions; e) plural extended pH sensitive degradable layers are disposed in the interior of the structural layer and at least two of the layers erode, dissolve or degrade at different pH triggers during use under physiological conditions; 0 plural extended pH sensitive degradable layers are present and at least two of those layers comprise different compositions; g) plural extended pH sensitive degradable layers are present and at least one of those layers comprises at least one functional excipient and at least one other of those layers excludes a functional excipient; h) plural extended pH sensitive degradable layers are present and one or more of those layers comprises at least one functional excipient and/or at least one active agent; i) plural extended pH sensitive degradable layers are present and at least one of those layers comprises at least one active and at least one other of those layers excludes an active agent; j) plural extended pH sensitive degradable layers are present and two or more of those layers comprise different functional excipient(s) and/or different active agent(s); or k) combinations thereof.

Some embodiments of the invention include those wherein: a) the device comprises, in the order from lumen to exterior of the device, an interior first extended pH sensitive degradable layer, an adjacent second extended pH sensitive degradable layer, an adjacent extended structural layer, an adjacent third extended pH sensitive degradable layer, and an adjacent exterior fourth extended pH sensitive degradable layer; b) the device comprises, in the order from lumen to exterior of the device, an interior first extended pH sensitive degradable layer, an adjacent extended structural layer, an adjacent second extended pH sensitive degradable layer, and an adjacent exterior third extended pH sensitive degradable layer; c) the device comprises, in the order from lumen to exterior of the device, an interior first extended pH sensitive degradable layer, an adjacent second extended pH sensitive degradable layer, an adjacent extended structural layer, and an adjacent exterior third extended pH sensitive degradable layer.

In some embodiments, the bacterial contamination is caused by Escherichia coli, Lactobacillus bacterium, Proteus bacterium, Pseudomonas aeruginosa, Proteus mirabilis, Enterococcus faecalis, Klebsiella pneumoniae, Staphylococcus aureus and Staphylococcus epidermidis or other such bacteria known to be capable of colonizing (contaminating) the urinary tract.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Preferred features and embodiments of each aspect of the invention are as for each of the other aspects mutatis mutandis unless context demands otherwise.

Embodiments of the present invention will now be described, by way of example only, with reference to the accompanying figures. The invention also includes all combinations and subcombinations of the aspects and embodiments disclosed herein.

### BRIEF DESCRIPTION OF THE FIGURES

The following figures form part of the present description and describe exemplary embodiments of the claimed invention. The skilled artisan will, in light of these figures and the description herein, be able to practice the invention without undue experimentation.

FIG. 1 illustrates the steps of bacterial colonisation of a catheter of the present invention wherein colonising bacteria illustrated by * begin to colonize the device following insertion (1), such that the surface becomes colonized (2), and a microbial biofilm is formed (3), the urinary pH is increased by Urea-splitting bacteria (4), and erosion of Eudragit® occurs at elevated pH leading to removal of biofilm and insoluble deposits (5).

FIG. 2 illustrates the drug eluting/self-cleansing layer (i) and the functional/structural layer imparting structural integrity to the device (ii) of a two layer system (a) and a three layer system (b).

FIG. 3 illustrates torque on the screw for a polymer that dissolves at pH 7 with a 5, 10 and 20% loading of the quinolone antibiotic Nalidixic Acid.

FIG. 4 illustrates mechanical properties of formulations can be examined using DMTA: or Dynamic Mechanical Thermal Analysis in tension mode.

FIG. 5 illustrates the release profile of 10% Nalidixic Acid from a device having a pH sensitive layer at pH 6 and pH 7, pH 6 taken to represent healthy uninfected urine.

FIG. 6 illustrates the release profile of 10% Nalidixic Acid from a device having a pH sensitive layer at pH 6 and pH 7.

FIG. 7 illustrates the release profile of antimicrobial Levofloxacin from a device having a pH sensitive layer of Eudragit® L100 comprising 5% Levofloxacin at pH 6.2 and pH 7.8.

FIG. 8 illustrates the release profile of antimicrobial Levofloxacin from a device having a pH sensitive layer of Eudragit® L100 comprising 10% Levofloxacin at pH 6.2 and pH 7.8.

FIG. 9 illustrates the release profile of antimicrobial Levofloxacin from three devices having a pH sensitive layer of Eudragit® L100 comprising 5%, 10% and 20% Levofloxacin respectively.

FIG. 10 illustrates the release profile of antimicrobial Levofloxacin from three devices having a pH sensitive layer of Eudragit® 4155F (powdered Eudragit® FS; anionic polymer based upon methyl acrylate, methyl methacrylate and methacrylic acid; dissolution above pH 7.0; poly(methy acrylate-co-methyl methacrylate-co-methacrylic acid) 7:3:1) comprising 5%, 10% and 20% Levofloxacin respectively.

FIG. 11 illustrates the release profile of antimicrobial Levofloxacin from a device having a pH sensitive layer of Eudragit® 4155F comprising 5% Levofloxacin at pH 6.2 and pH 7.8.

FIG. 12 illustrates the release profile of antimicrobial Levofloxacin from a device having a pH sensitive layer of Eudragit® 4155F comprising 10% Levofloxacin at pH 6.2 and pH 7.8.

FIG. 13 illustrates the release profile of antimicrobial Levofloxacin from a device having a pH sensitive layer of Eudragit® 4155F comprising 20% Levofloxacin at pH 6.2 and pH 7.8.

FIG. 14 illustrates the release profile of antimicrobial Levofloxacin from a first device having a pH sensitive layer of Eudragit® 4155F and a second device having pH sensitive layer of Eudragit® L100 at pH 6.2 for 2 hours, pH 7.8 for 2 hours and pH 6.2 for 2 hours.

FIG. 15 illustrates the release profile of antimicrobial Levofloxacin from a first device having a pH sensitive layer of Eudragit® 4155F and a second device having pH sensitive layer of Eudragit® L100 at a pH of 7.8 for 2 hours, pH 6.2 for 2 hours and pH 7.8 for 2 hours.

FIG. 16 illustrates mean percentage mass over time at pH 6.2 for a first device having a pH sensitive layer of Eudragit® 4155F comprising 10% CA and a second device having a pH sensitive layer of Eudragit® 4155F comprising 10% CA and 10% Nalidixic acid.

FIG. 17 illustrates mean percentage mass over time at pH 6.2 for a first device having a pH sensitive layer of Eudragit® L100, a second device having a pH sensitive layer of Eudragit® L100 comprising 10% Nalidixic acid and a third device having a pH sensitive layer of Eudragit® L100 comprising 10% Levofloxacin.

FIG. 18 illustrates mean percentage mass over time at pH 6.2 for a first device having a pH sensitive layer of Eudragit® L100, a second device having a pH sensitive layer of Eudragit® L100 comprising 10% Nalidixic acid and a third device having a pH sensitive layer of Eudragit® L100 comprising 10% Levofloxacin.

FIG. 19 illustrates mean percentage mass over time at pH 6.2 for a first device having a pH sensitive layer of Eudragit® 4155F, a second device having a pH sensitive layer of Eudragit® 4155F comprising 10% Nalidixic acid and a third device having a pH sensitive layer of Eudragit® 4155F comprising 10% Levofloxacin.

FIG. 20 illustrates the increased bacterial adherence of PMIR to a first device formed from PVC after 4 hours immersion in artificial urine compared to a second device having a pH sensitive layer of Eudragit® 4155F after 4 hours immersion in artificial urine.

FIGS. 21A-21B depict exemplary embodiments of a bi-layered device according to the invention.

FIG. 22 depicts an exemplary embodiment of a tri-layered device according to the invention.

FIGS. 23A-23B depict exemplary embodiments of a 4-layered device according to the invention.

FIG. 24 depicts an exemplary embodiment of a 5-layered device according to the invention.

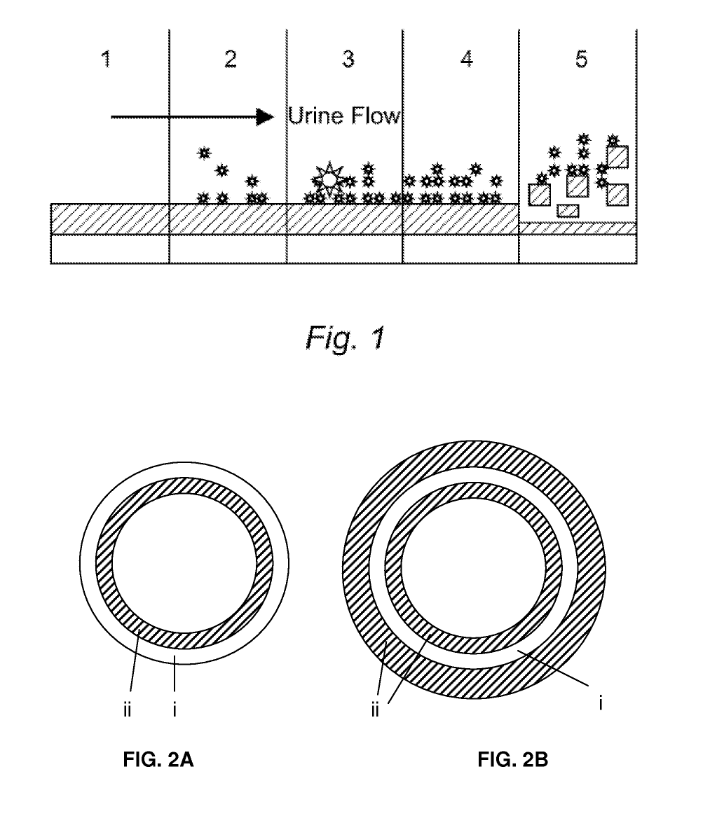

### DETAILED DESCRIPTION

Polymers were mixed with suitable plasticizers to enable processing with a twin-screw extruder. Different drug loadings of different antibacterial agents were then mixed with the polymer/plasticizer formulations. Formulations were stored in a dessicator for 24 hours prior to processing. The formulations were then extruded with varying concentrations of antibacterial agents. The samples were suspended in release medium appropriate to the in-vivo conditions. Samples were then filtered using 0.45 μm syringe filters and analysed using UV spectroscopy to determine their drug release properties.

It is expected that single drug loaded Eudragit® films will be manufactured using a twin-screw extrusion system that possess the ability to feed the antimicrobial, EDTA and citric acid at four different ports along the extruder barrel. This coupled with the modular design of the screw will allow products with extremely uniform density and homogeneity to be produced without degradation of the functional excipients and/or active agent(s).

Once manufactured, characterization and selection of optimized film layers for co-extrusion with PVC can be undertaken. Multi-layer extrusion of PVC and the optimised pH responsive layers can be performed on state-of-the-art multi-layer sheet extrusion facilities. Whilst typical urinary devices tend to be tubular, multi-layered sheets will be extruded to allow for testing.

Prior to co-extrusion, drug loaded Eudragit® pellets can be prepared using an air-cooled die face pelletiser connected to a twin-screw kneader. These pellets can be used to investigate the effects of plasticizer type, plasticizer content and the effects of the inclusion of other functional excipients (EDTA, citric acid, Chlorhexidine and its salts, nalidixic acid) on the rheological properties of the Eudragit® polymers; which must be carefully controlled to optimize the operating temperatures of the co-extrusion process and the final properties of the film.

Once optimized processing conditions have been determined using knowledge gained from mDSC and thermorheological experiments, systems in which multi-layered films of varying layer thickness can be manufactured.

The multi-layered tubes/devices of the invention can be constructed according to many different embodiments. Exemplary structural embodiments are depicted in FIGS. 2A (2-layered), 2B (3-layered), 21A (2-layered), 21B (2-layered), 22 (3-layered), 23A (4-layered), 23B (4-layered) and 24 (5-layered).

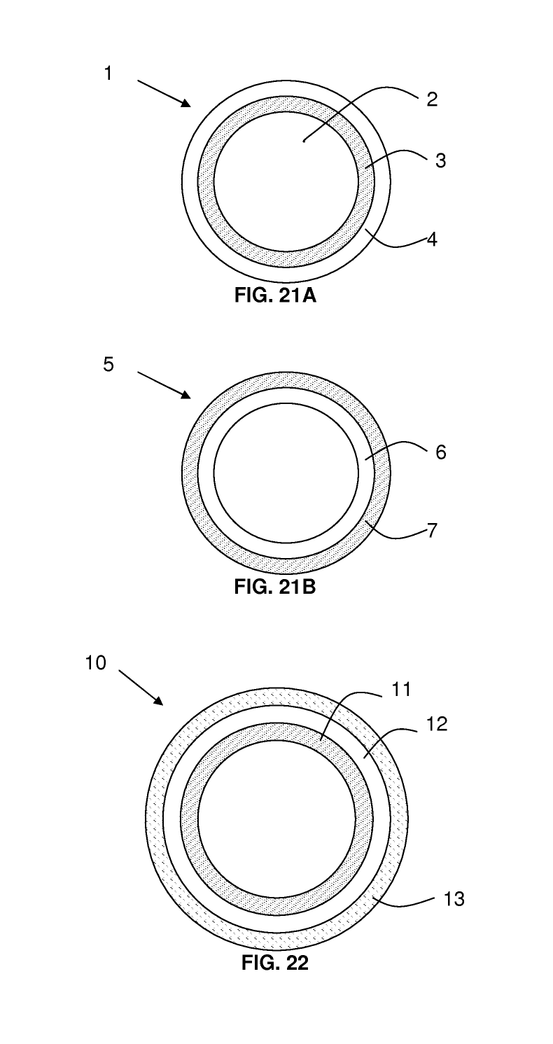

Extended device (1) depicted in FIG. 21A comprises an interior layer (3) defining a lumen (2) and a coextensive exterior layer (4) immediately adjacent to the interior layer. Various different embodiments of this device are contemplated as detailed in Table 1A and the related description below, wherein exemplary compositions of the different layers are described. It should be noted that the terms “pH sensitive layer” and “erodible layer” are used interchangeably herein.

TABLE 1A
(Embodiment of FIG. 21A. Suitable for use as urinary catheter.)

| | General | |
|---|---|---|
| Layer | Description | Properties and Composition |
| 3 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 4 | Exterior | Layer maintains structural integrity and does not erode, |
| | structural layer | dissolve or degrade during use. |
| | (non-pH | |
| | sensitive) | |

The pH sensitive layer (3) can comprise cellulose-based enteric polymers or anionic methyl methacrylate copolymers. Exemplary materials include HPMC-AS (HF grade), Eudragit® FS30D and Eudragit® 5100. Also Eudragit® L100, Eudragit® L100-55, HPMC-AS (LF and MF grades), CAP (cellulose acetate phthalate) and CAT (cellulose acetate trimellitate). An organic acid (present in an amount of about 1-50% wt. or 5-30% wt. of the layer) can be added to these polymeric materials to control the erosion/dissolution rate/pH (even the drug release rate) of a corresponding layer. The higher the organic acid content, the slower the corresponding polymer/layer will dissolve/erode in an aqueous environment and even more so as the pH of the environment increases, such as to exceed pH 6.5 or pH 7. In general, higher organic acid content will reduce the microenvironment pH and thus retard erosion of polymer layer and release rate of active.

The structural layer (4) can comprise silicone, latex, PVC (poly (vinyl chloride)), polyurethane, ethylene-vinylacetate copolymer, polyethylene, polypropylene, polyester, polystyrene, nylon, or a combination thereof. The structural layer is preferably flexible/pliable.

In some embodiments, the erodible layer (3) excludes an active agent and the structural layer (4) comprises an active agent and provides a continuous diffusion-based release of drug to the local environment of use. Exemplary active agents include levofloxacin, chlorhexidine, nalidixic acid, rifampicin and salicylic acid.

In some embodiments, the structural layer (4) excludes an active agent and the erodible layer (3) comprises an active agent and provides a continuous diffusion-based release of drug to the local environment of use, wherein the release of drug at pH 6 is slow and the release rate of drug increases as the pH of the environment of use begins to approach and exceeds pH 7.

In some embodiments, the structural layer (4) excludes an active agent and the erodible layer (3) excludes an active agent. In some embodiments, the structural layer (4) comprises an active agent, the erodible layer (3) comprises the same or a different active agent, and the active agent is released as described herein for each respective layer.

Extended device (5) depicted in FIG. 21B comprises an interior layer (6) defining a lumen and a coextensive exterior layer (7) immediately adjacent to the interior layer. Various different embodiments of this device are contemplated as detailed in Table 1B and the related description below, wherein exemplary compositions of the different layers are described.

TABLE 1B
(Embodiment of FIG. 21B. Suitable for use as urinary catheter or a ureteral stent.)

| | General | |
|---|---|---|
| Layer | Description | Properties and Composition |
| 7 | Exterior pH | Dissolution, erosion or degradation pH trigger occurs at pH |
| | sensitive layer | 7. A surface erodible layer that begins to erode when |
| | | exposed to an aqueous environment having a pH value ≧7. |
| 6 | Interior | Layer maintains structural integrity and does not erode, |
| | structural layer | dissolve or degrade during use. |
| | (non-pH | |
| | sensitive) | |
| | defining the | |
| | lumen | |

The extended device (5) comprises a structure that is substantially the inverted structure of the extended device (1). The compositions of the structural layer (6) can be selected from the same compositions as for the structural layer (4). The compositions of the erodible layer (7) can be selected from the same compositions as for the erodible layer (3).

In some embodiments, the erodible layer (7) excludes an active agent and the structural layer (6) comprises an active agent and provides a continuous diffusion-based release of drug to the local environment of use.

In some embodiments, the structural layer (6) excludes an active agent and the erodible layer (7) comprises an active agent and provides a continuous diffusion-based release of drug to the local environment of use, wherein the release of drug at pH 6 is slow and the release rate of drug increases as the pH of the environment of use begins to approach and exceeds pH 7.

In some embodiments, the structural layer (6) excludes an active agent and the erodible layer (7) excludes an active agent. In some embodiments, the structural layer (6) comprises an active agent, the erodible layer (7) comprises the same or a different active agent, and the active agent is released as described herein for each respective layer.

Extended device (10) depicted in FIG. 22 comprises an interior layer (11) defining a lumen, a coextensive intermediate layer (12) immediately adjacent the interior layer, and a coextensive exterior layer (13) immediately adjacent the intermediate layer (12). Various different embodiments of this device are contemplated as detailed in Tables 2A-2D and the related description below, wherein exemplary compositions of the different layers are described.

TABLE 2A
(Embodiment of FIG. 22. Suitable for use as urinary catheter or a ureteral stent.)

| | General | |
|---|---|---|
| Layer | Description | Properties and Composition |
| 11 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 12 | Structural layer | Layer maintains structural integrity and does not erode, |
| | (non-pH | dissolve or degrade during use. |
| | sensitive) | |
| 13 | Exterior pH | Dissolution, erosion or degradation pH trigger occurs at pH |
| | sensitive layer | 7. A surface erodible layer that begins to erode when |
| | | exposed to an aqueous environment having a pH value ≧7. |

Even though the layers (11) and (13) can have substantially the same performance properties, their compositions can be the same or different. The compositions of the structural layer (12) can be selected from the same compositions as for the structural layer (4). The compositions of the erodible layers (11) and (13) can be selected from the same compositions as for the erodible layer (3).

The layers (11, 12, 13) of the extended device (10) independently comprise or exclude an active agent upon each occurrence, meaning that one, two or all three layers can comprise an active agent, and the active agent present in one layer can be independently the same as or different than the active agent in another layer. In some embodiments, the structural layer (12) excludes an active agent and both erodible layers (11, 13) exclude an active agent. In some embodiments, the structural layer (12) comprises an active agent, the erodible layer (11) comprises the same or a different active agent, the erodible layer (13) comprises the same or a different active agent, and the active agent is released as described herein for each respective layer. In some embodiments, the structural layer (12) excludes an active agent, the erodible layer (11) comprises an active agent, the erodible layer (13) comprises the same or a different active agent, and the active agent is released as described herein for each respective layer. In some embodiments, the structural layer (12) excludes an active agent, the erodible layer (11) excludes an active agent, the erodible layer (13) comprises an active agent, and the active agent is released as described herein. In some embodiments, the structural layer (12) excludes an active agent, the erodible layer (11) comprises an active agent, the erodible layer (13) excludes an active agent, and the active agent is released as described herein.

TABLE 2B
(Alternate embodiment of FIG. 22. Suitable for
use as urinary catheter or a ureteral stent.)

| | General | |
|---|---|---|
| Layer | Description | Properties and Composition |
| 11 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 12 | Structural layer | Layer maintains structural integrity and does not erode, |
| | (non-pH | dissolve or degrade during use. |
| | sensitive) | |
| 13 | Exterior Ph | Dissolution, erosion or degradation pH trigger occurs at pH |
| | sensitive layer | 6. A surface erodible layer that begins to erode when |
| | | exposed to an aqueous environment having a pH value ≧6. |

A substantial difference between the device of Table 2A and that of Table 2B is the difference in dissolution/erosion pH (trigger pH) of the exterior layer (13). Polymeric materials suitable for making the layer with trigger pH of 6 include cellulose-based enteric polymers, anionic copolymers based on methyl acrylate, methyl methacrylate and methacrylic acid. Exemplary polymers include HPMC-AS (MF and LF grades), Eudragit® L100, Eudragit® L100-55, CAP and CAT. Small organic acids (1-50%) can be used to tailor erosion/release rate.

In the case of layers with a trigger pH of 6, polymers such as HPMC-AS (MF, LF), CAP, CAT, Eudragit® L100 and Eudragit® L100-55 may be used. The incorporation of organic acids will reduce erosion when the pH of the surrounding fluid exceeds 6. Eudragit® L100 and HPMC-AS (MF) do not require the use of organic acids to erode at pH values exceeding (≧) 6. At lower pH values they will remain intact. In regard to CAP, CAT, HPMC-AS (LF) and L100-55, some embodiments comprise (1-50% wt.) organic acids to ensure that they do not degrade at pH values <6. Although such examples may be beneficial they will last in the environment of use only as long as organic acid is present. Eudragit® L100 and HPMC-AS (MF) are the most suitable polymers for pH 6 trigger.

TABLE 2C
(Alternate embodiment of FIG. 22. Suitable for
use as urinary catheter or a ureteral stent.)

| | General | |
|---|---|---|
| Layer | Description | Properties and Composition |
| 11 | Interior pH | Dissolution, erosion or degradation pH trigger occurs at pH |
| | sensitive layer | 6. A surface erodible layer that begins to erode when |
| | defining the | exposed to an aqueous environment having a pH value ≧6. |
| | lumen | |
| 12 | Structural layer | Layer maintains structural integrity and does not erode, |
| | (non-pH | dissolve or degrade during use. |
| | sensitive) | |
| 13 | Exterior Ph | Dissolution, erosion or degradation pH trigger occurs at pH |
| | sensitive layer | 7. A surface erodible layer that begins to erode when |
| | | exposed to an aqueous environment having a pH value ≧7. |

The alternate embodiment of Table 2C is essentially an inversion of the structure of the embodiment of Table 2A. Accordingly, the substantial differences between the device of Table 2A and that of Table 2C are the differences in dissolution/erosion pH (trigger pH) of the exterior layer (13) and the interior layer (11).

TABLE 2D
(Alternate embodiment of FIG. 22. Suitable for
use as urinary catheter or a ureteral stent.)

| | General | |
|---|---|---|
| Layer | Description | Properties and Composition |
| 11 | Interior pH | Dissolution, erosion or degradation pH trigger occurs at pH |
| | sensitive layer | 6. A surface erodible layer that begins to erode when |
| | defining the | exposed to an aqueous environment having a pH value ≧6. |
| | lumen | |
| 12 | Structural layer | Layer maintains structural integrity and does not erode, |
| | (non-pH | dissolve or degrade during use. |
| | sensitive) | |
| 13 | Exterior Ph | Dissolution, erosion or degradation pH trigger occurs at pH |
| | sensitive layer | 6. A surface erodible layer that begins to erode when |
| | | exposed to an aqueous environment having a pH value ≧6. |

The alternate embodiment of Table 2D is essentially a change in the composition of the interior and exterior erodible layers (11, 13), wherein both layers of the embodiment of Table 2A have a trigger pH of 7; whereas, both layers of the embodiment of Table 2D have a trigger pH of 6.

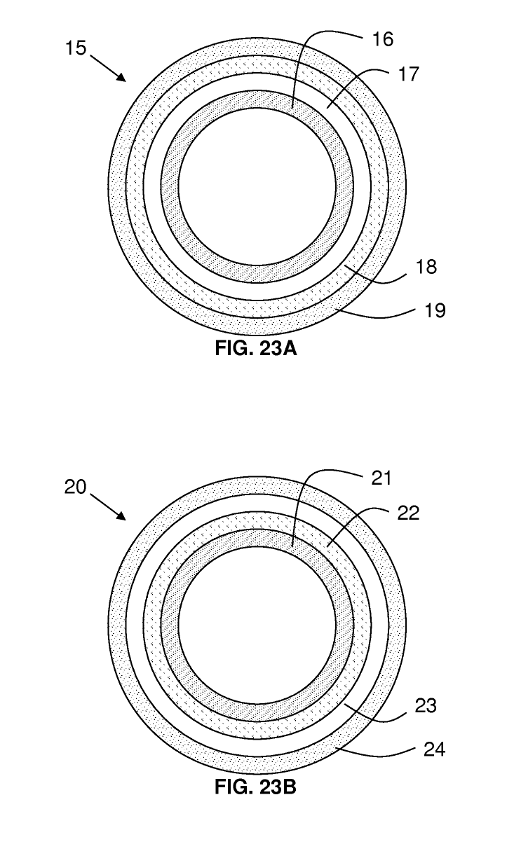

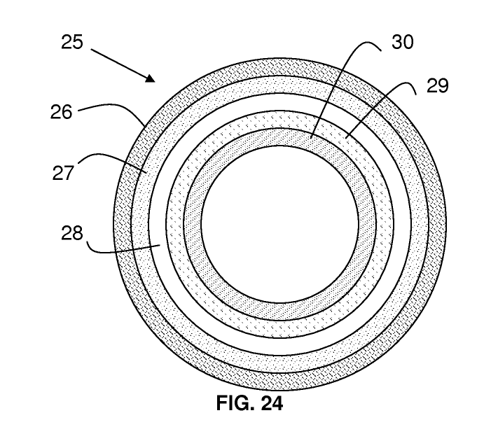

Extended device (15) depicted in FIG. 23A comprises an interior layer (16) defining a lumen, a coextensive first intermediate layer (17) immediately adjacent the interior layer, a coextensive second intermediate later (18) immediately adjacent the first intermediate layer and a coextensive exterior layer (19) immediately adjacent the second intermediate layer (18). Various different embodiments of this device are contemplated as detailed in Tables 3A-3B and the related description below, wherein exemplary compositions of the different layers are described.

TABLE 3A
(Embodiment of FIG. 23A. Suitable for use as urinary catheter or ureteral stent.)

| | General | |
|---|---|---|
| Layer | Description | Properties and Composition |
| 16 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 17 | Structural layer | Layer maintains structural integrity and does not erode, |
| | (non-pH | dissolve or degrade during use. |
| | sensitive) | |
| 18 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 19 | Exterior pH | Dissolution, erosion or degradation pH trigger occurs at pH |
| | sensitive layer | 7. A surface erodible layer that begins to erode when |
| | | exposed to an aqueous environment having a pH value ≧7. |

Even though the layers (16, 18, 19) can have substantially the same performance properties, their compositions can be the same or different. The compositions of the structural layer (17) can be selected from the same compositions as for the structural layer (4). The compositions of the erodible layers (16, 18, 19) can be selected from the same compositions as for the erodible layer (3).

The layers (16, 17, 18, 19) of the extended device (15) independently comprise or exclude an active agent upon each occurrence, meaning that one, two, three or all fours layers can comprise an active agent, and the active agent present in one layer can be independently the same as or different than the active agent in another layer. In some embodiments, all layers (16, 17, 18, 19) comprise an active agent. In some embodiments, all layers (16, 17, 18, 19) exclude an active agent. In some embodiments, the structural layer (17) excludes an active agent and all three erodible layers (16, 18, 19) comprise an active agent. In some embodiments, the structural layer (17) and the second intermediate erodible layer (18) both exclude an active agent, the interior erodible layer (16) comprises an active agent, the exterior erodible layer (19) comprises the same or a different active agent, and the active agent is released as described herein for each respective layer. In some embodiments, the structural layer (17), the second intermediate erodible layer (18) and the interior erodible layer (16) all exclude an active agent, the exterior erodible layer (19) comprises an active agent, and the active agent is released as described herein.

TABLE 3B
(Alternate embodiment of FIG. 23A. Suitable for
use as urinary catheter or ureteral stent.)

| | General | |
|---|---|---|
| Layer | Description | Properties and Composition |
| 16 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 17 | Structural layer | Layer maintains structural integrity and does not erode, |
| | (non-pH | dissolve or degrade during use. |
| | sensitive) | |
| 18 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 19 | Exterior pH | Dissolution, erosion or degradation pH trigger occurs at pH |
| | sensitive layer | 6. A surface erodible layer that begins to erode when |
| | | exposed to an aqueous environment having a pH value ≧6. |

The alternate embodiment of Table 3B is similar to that of Table 3A with the exception that the exterior erodible layer of Table 3B is adapted to begin dissolution or erosion at a trigger pH of ≧6.

Extended device (20) depicted in FIG. 23B comprises an interior layer (21) defining a lumen, a coextensive first intermediate layer (22) immediately adjacent the interior layer, a coextensive second intermediate later (23) immediately adjacent the first intermediate layer and a coextensive exterior layer (24) immediately adjacent the second intermediate layer (23). The device (20) is similar in construction to the device (15) with the exception that the order of the two intermediate layers is reversed. Various different embodiments of this device are contemplated as detailed in Tables 4A-4B and the related description below, wherein exemplary compositions of the different layers are described.

TABLE 4A
(Embodiment of FIG. 23B. Suitable for use as urinary catheter or ureteral stent.)

| | General | |
|---|---|---|
| Layer | Description | Properties and Composition |
| 21 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 22 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 23 | Structural layer | Layer maintains structural integrity and does not erode, |
| | (non-pH | dissolve or degrade during use. |
| | sensitive) | |
| 24 | Exterior pH | Dissolution, erosion or degradation pH trigger occurs at pH |
| | sensitive layer | 7. A surface erodible layer that begins to erode when |
| | | exposed to an aqueous environment having a pH value ≧7. |

Even though the layers (21, 22, 24) can have substantially the same performance properties, their compositions can be the same or different. The compositions of the structural layer (23) can be selected from the same compositions as for the structural layer (4). The compositions of the erodible layers (21, 22, 24) can be selected from the same compositions as for the erodible layer (3).

The layers (21, 22, 23, 24) of the extended device (20) independently comprise or exclude an active agent upon each occurrence, meaning that one, two, three or all fours layers can comprise an active agent, and the active agent present in one layer can be independently the same as or different than the active agent in another layer. In some embodiments, all layers (21, 22, 23, 24) comprise an active agent. In some embodiments, all layers (21, 22, 23, 24) exclude an active agent. In some embodiments, the structural layer (23) excludes an active agent and all three erodible layers (21, 22, 24) comprise an active agent. In some embodiments, the structural layer (23) and the second intermediate erodible layer (22) both exclude an active agent, the interior erodible layer (21) comprises an active agent, the exterior erodible layer (24) comprises the same or a different active agent, and the active agent is released as described herein for each respective layer. In some embodiments, the structural layer (23), the second intermediate erodible layer (22) and the interior erodible layer (21) all exclude an active agent, the exterior erodible layer (24) comprises an active agent, and the active agent is released as described herein.

TABLE 4B
(Alternate embodiment of FIG. 23B. Suitable for
use as urinary catheter or ureteral stent.)

| | General | |
|---|---|---|
| Layer | Description | Properties and Composition |
| 21 | Interior pH | Dissolution, erosion or degradation pH trigger occurs at pH |
| | sensitive layer | 6. A surface erodible layer that begins to erode when |
| | defining the | exposed to an aqueous environment having a pH value ≧6. |
| | lumen | |
| 22 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 23 | Structural layer | Layer maintains structural integrity and does not erode, |
| | (non-pH | dissolve or degrade during use. |
| | sensitive) | |
| 24 | Exterior pH | Dissolution, erosion or degradation pH trigger occurs at pH |
| | sensitive layer | 7. A surface erodible layer that begins to erode when |
| | | exposed to an aqueous environment having a pH value ≧7. |

The alternate embodiment of Table 4B is similar to that of Table 4A with the exception that the interior erodible layer of Table 4B is adapted to begin dissolution or erosion at a trigger pH ≧6.

Extended device (25) depicted in FIG. 24 comprises an interior layer (30) defining a lumen, a coextensive first intermediate layer (29) immediately adjacent the interior layer, a coextensive second intermediate later (28) immediately adjacent the first intermediate layer, a coextensive third intermediate layer (27) adjacent the second intermediate layer, and a coextensive exterior layer (26) immediately adjacent the third intermediate layer (27). Various different embodiments of this device are contemplated as detailed in Tables 5A-5D and the related description below, wherein exemplary compositions of the different layers are described.

TABLE 5A
(Embodiment of FIG. 24. Suitable for use as urinary catheter or ureteral stent.)

| | General | |
|---|---|---|
| Layer | Description | Properties and Composition |
| 30 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 29 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 28 | Structural layer | Layer maintains structural integrity and does not erode, |
| | (non-pH | dissolve or degrade during use. |
| | sensitive) | |
| 27 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 26 | Exterior pH | Dissolution, erosion or degradation pH trigger occurs at pH |
| | sensitive layer | 7. A surface erodible layer that begins to erode when |
| | | exposed to an aqueous environment having a pH value ≧7. |

Even though the layers (26, 27, 29, 30) can have substantially the same performance properties, their compositions can be the same or different. The compositions of the structural layer (28) can be selected from the same compositions as for the structural layer (4). The compositions of the erodible layers (26, 27, 29, 30) can be selected from the same compositions as for the erodible layer (3).

The layers (26, 27, 28, 29, 30) of the extended device (25) independently comprise or exclude an active agent upon each occurrence, meaning that one, two, three, fours or all five layers can comprise an active agent, and the active agent present in one layer can be independently the same as or different than the active agent in another layer. In some embodiments, all layers (26, 27, 28, 29, 30) comprise an active agent. In some embodiments, all layers (26, 27, 28, 29, 30) exclude an active agent. In some embodiments, the structural layer (23) excludes an active agent and all four erodible layers (26, 27, 29, 30) comprise an active agent. In some embodiments, the structural layer (28) and the first intermediate erodible layer (29) both exclude an active agent, the interior erodible layer (30) comprises an active agent, the exterior erodible layer (26) comprises the same or a different active agent, the second intermediate erodible layer comprises an active agent, and the active agent is released as described herein for each respective layer. In some embodiments, the structural layer (28), the second intermediate erodible layer (27) and the first intermediate erodible layer (29) all exclude an active agent, the exterior erodible layer (26) comprises an active agent, the interior erodible layer (30) comprises an active agent, and the active agent is released as described herein.

TABLE 5B
(Alternate embodiment of FIG. 24. Suitable for
use as urinary catheter or ureteral stent.)

| | General | |
|---|---|---|
| Layer | Description | Properties and Composition |
| 30 | Interior pH | Dissolution, erosion or degradation pH trigger occurs at pH |
| | sensitive layer | 6. A surface erodible layer that begins to erode when |
| | defining the | exposed to an aqueous environment having a pH value ≧6. |
| | lumen | |
| 29 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 28 | Structural layer | Layer maintains structural integrity and does not erode, |
| | (non-pH | dissolve or degrade during use. |
| | sensitive) | |
| 27 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 26 | Exterior pH | Dissolution, erosion or degradation pH trigger occurs at pH |
| | sensitive layer | 7. A surface erodible layer that begins to erode when |
| | | exposed to an aqueous environment having a pH value ≧7. |

The alternate embodiment of Table 5B is similar to that of Table 5A with the exception that the interior erodible layer of Table 5B is adapted to begin dissolution or erosion at a trigger pH of ≧6.

TABLE 5C
(Alternate embodiment of FIG. 24. Suitable for
use as urinary catheter or ureteral stent.)

| | General | |
|---|---|---|
| Layer | Description | Properties and Composition |
| 30 | Interior pH | Dissolution, erosion or degradation pH trigger occurs at pH |
| | sensitive layer | 7. A surface erodible layer that begins to erode when |
| | defining the | exposed to an aqueous environment having a pH value ≧7. |
| | lumen | |
| 29 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 28 | Structural layer | Layer maintains structural integrity and does not erode, |
| | (non-pH | dissolve or degrade during use. |
| | sensitive) | |
| 27 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 26 | Exterior pH | Dissolution, erosion or degradation pH trigger occurs at pH |
| | sensitive layer | 6. A surface erodible layer that begins to erode when |
| | | exposed to an aqueous environment having a pH value ≧6. |

The alternate embodiment of Table 5C is similar to that of Table 5A with the exception that the exterior erodible layer of Table 5C is adapted to begin dissolution or erosion at a trigger pH of ≧6.

TABLE 5D
(Alternate embodiment of FIG. 24. Suitable for
use as urinary catheter or ureteral stent.)

| | General | |
|---|---|---|
| Layer | Description | Properties and Composition |
| 30 | Interior pH | Dissolution, erosion or degradation pH trigger occurs at pH |
| | sensitive layer | 6. A surface erodible layer that begins to erode when |
| | defining the | exposed to an aqueous environment having a pH value ≧6. |
| | lumen | |
| 29 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 28 | Structural layer | Layer maintains structural integrity and does not erode, |
| | (non-pH | dissolve or degrade during use. |
| | sensitive) | |
| 27 | Intermediate pH | Dissolution, erosion or degradation pH trigger occurs at pH |
| | sensitive layer | 7. A surface erodible layer that begins to erode when |
| | | exposed to an aqueous environment having a pH value ≧7. |
| 26 | Exterior pH | Dissolution, erosion or degradation pH trigger occurs at pH |
| | sensitive layer | 6. A surface erodible layer that begins to erode when |
| | | exposed to an aqueous environment having a pH value ≧6. |

The alternate embodiment of Table 5D is similar to that of Table 5A with the exception that the exterior erodible layer and the exterior erodible layer of Table 5D are both adapted to begin dissolution or erosion at a trigger pH of ≧6.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

In view of the above description and the examples below, one of ordinary skill in the art will be able to practice the invention as claimed without undue experimentation. The foregoing will be better understood with reference to the following examples that detail certain procedures for the preparation of embodiments of the present invention. All references made to these examples are for the purposes of illustration. The following examples should not be considered exhaustive, but merely illustrative of only a few of the many embodiments contemplated by the present invention.

### Example 1

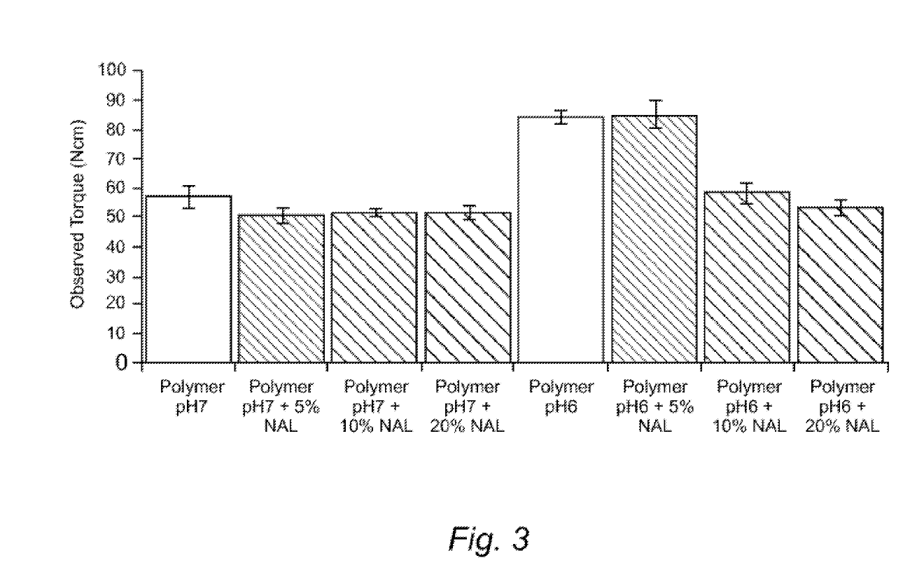

As illustrated by FIG. 3, the torque on the screw can be measured which provides a good indication of the viscosity and fluidity of the material within the extruder and gives an approximation of how different additives and functional excipients and/or active agents will affect both the ease of production of the material. This also has some bearing on the final mechanical properties of the material. This graph shows a polymer that dissolves at pH 7 with a 5, 10 and 20% loading of the quinolone antibiotic Nalidixic Acid. There was little effect on the torque with increasing nalidixic acid content. However, one of the other agents, levofloxacin, showed an increase in the observed torque, showing that it made processing more difficult.

When increasing levels of nalidixic acid were added to a pH 6 dissolving polymer, there was a decrease in the torque observed on the screw, indicating that with this polymer, the drug was aiding the processing.

### Example 2

Mechanical properties of formulations can be examined using DMTA: or Dynamic Mechanical Thermal Analysis in tension mode. This involves heating the product along a temperature gradient whilst constantly oscillating it around a set point. From this data, it is possible to determine the glass transition temperature which is the temperature below which the material exists as a glassy state and above which it exists in a more flexible, rubbery state. This provides an understanding of relaxation properties of the polymer, which will have implications on the flexibility of the final product.

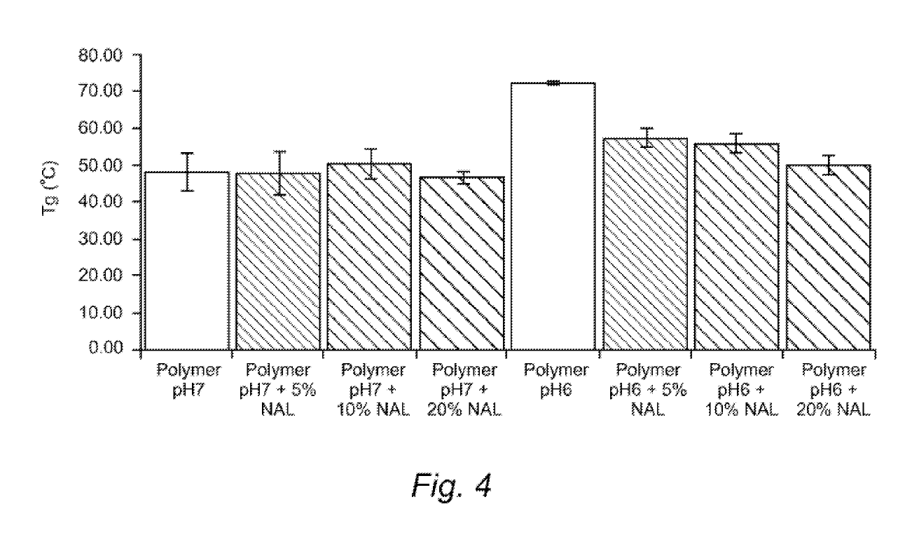

FIG. 4 illustrates values which reflect those observed during processing, with Nalidixic acid causing a decrease in the glass transition temperature with the pH 6 polymer. As before, the agent which had increased the torque during processing also increased the glass transition temperature.

Using strips of extruded formulations and examining them using DMTA in tension mode and complimenting the torque values, it was observed that levofloxacin increased the glass transition temperature observed, perhaps having an antiplasticization effect.

### Example 3

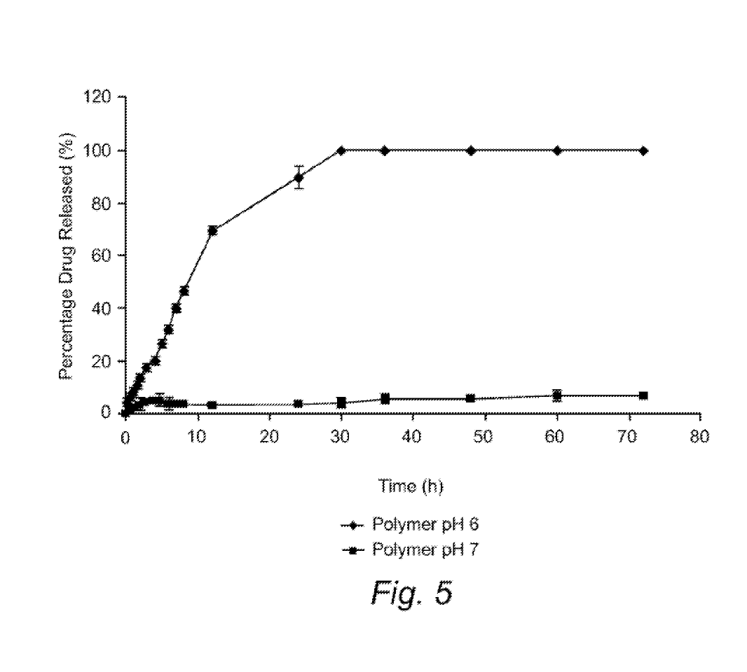

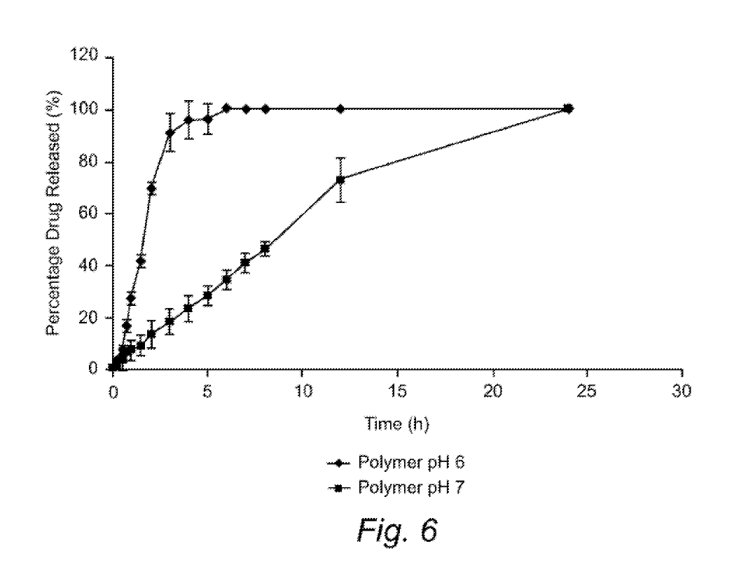

An embodiment of a particular formulation of the invention which could be used to form a particular layer of a device of the present invention was tested to determine drug elution characteristics at pH 6.2. Using the formulation, a layer could be provided which constantly elutes a drug at a low level when the device is in place, but, as a failsafe device, would have the ability to switch to a more rapid response when infection is detected that is, when the pH rises. Typically, pH 6.2 is the pH of healthy, uninfected urine, whilst pH 7.8 is the pH of infected urine. As illustrated in FIG. 5, drug release studies of 10% Nalidixic Acid were performed using dissolution apparatus with PBS solution at pH 6.2, to represent healthy urine, and pH 7.8 to represent infected urine.

Formulations used in this testing included a first formulation comprising Eudragit® S 100 and 10% PEG 8000 and a second formulation comprising Eudragit® L100 and 20 glycerol and 20% PEG 8000.

### Example 4

Embodiments of a particular formulation of the invention which could be used to form a particular layer of a device of the present invention were tested in a pH 7.8 medium, representing the infected urine. Formulations used in this testing included a first formulation comprising Eudragit® S100 and 10% PEG 8000 and a second formulation comprising Eudragit® L100 and 20 glycerol and 20% PEG 8000.

In comparison to the pH 6 dissolving polymer formulation discussed in Example 3, the pH 7 dissolving polymer releases its drug over a longer period of time.

The pH 6 dissolving polymer shows a much different release profile to the pH 7 polymer, allowing for a rapid response to the presence of infection, whilst the pH 7 dissolving polymer creates a protective barrier at the low pH values.

Although the invention has been particularly shown and described with reference to particular examples, it will be understood by those skilled in the art that various changes in the form and details may be made therein without departing from the scope of the present invention.

### Example 5

A first device comprising a pH sensitive layer including Eudragit® L100, and a second device comprising a pH sensitive layer including Eudragit® 4155F were formed. These devices allowed controlled release of the antimicrobial agents Levofloxacin and Nalidixic acid at physiological pH (approximately 6.2), and an enhanced release rate of these active agents at elevated pH levels generally associated with urinary infection (approximately 7.8).

In addition to providing a burst of antimicrobial the multi-layered films will also provide a new/clean surface that will be free from bacterial adherence.

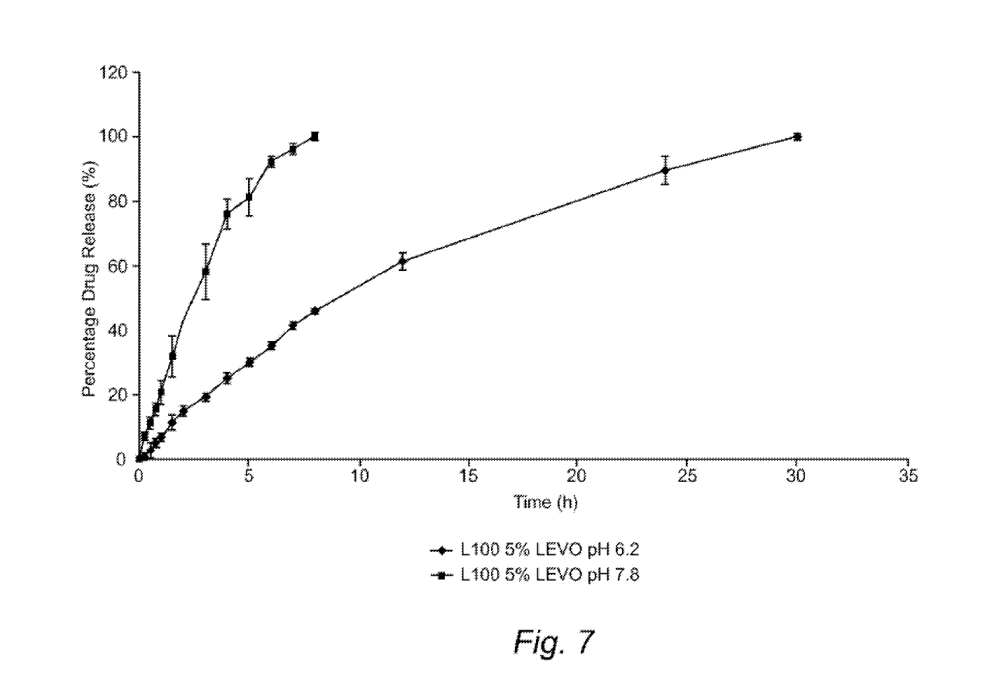

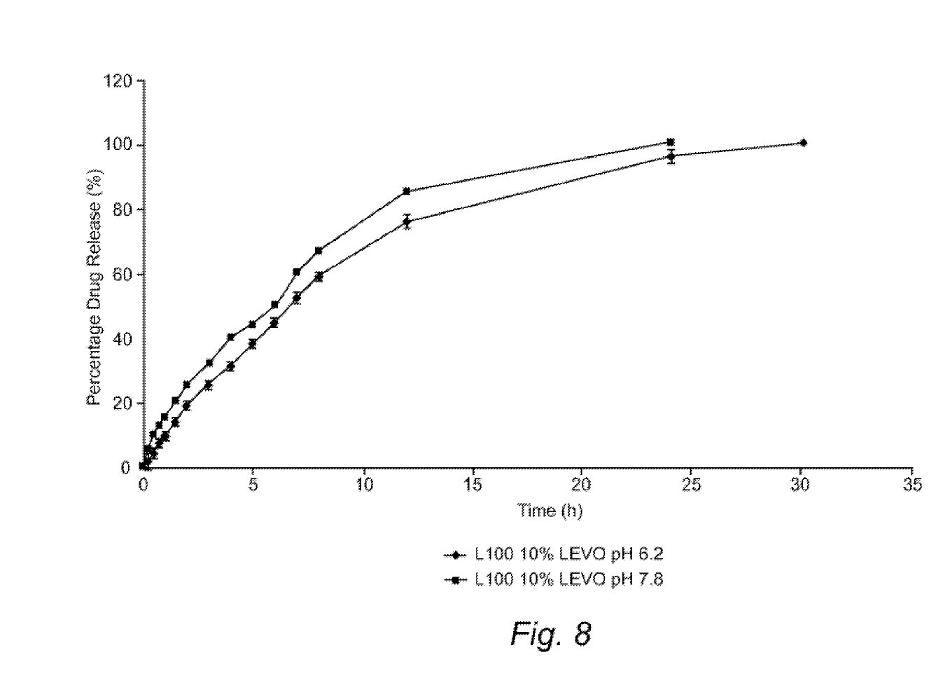

FIGS. 7 and 8 evidence that the release of antimicrobial from the devices can be modified by varying the pH of the release media and additionally through variation of antimicrobial loading.

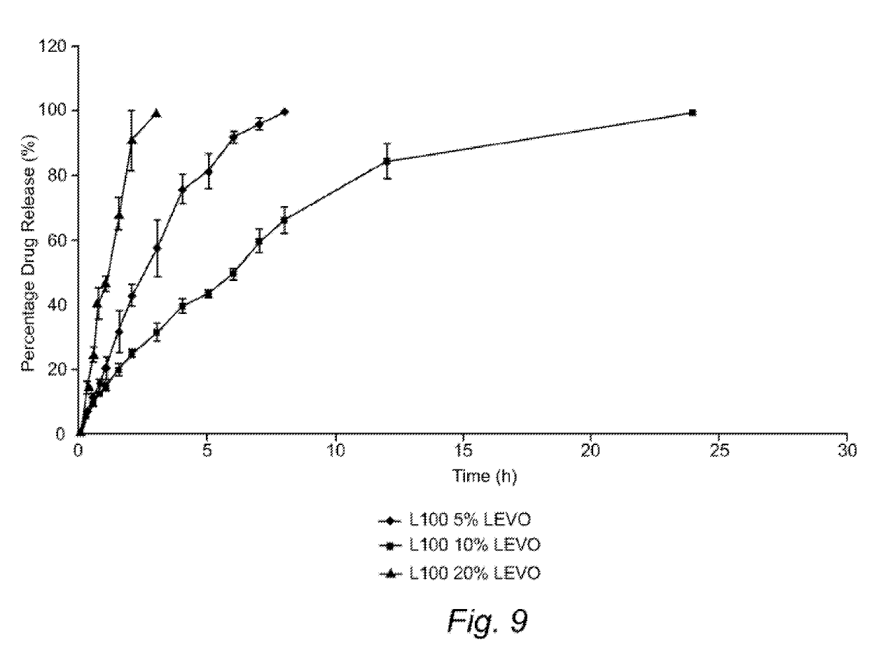

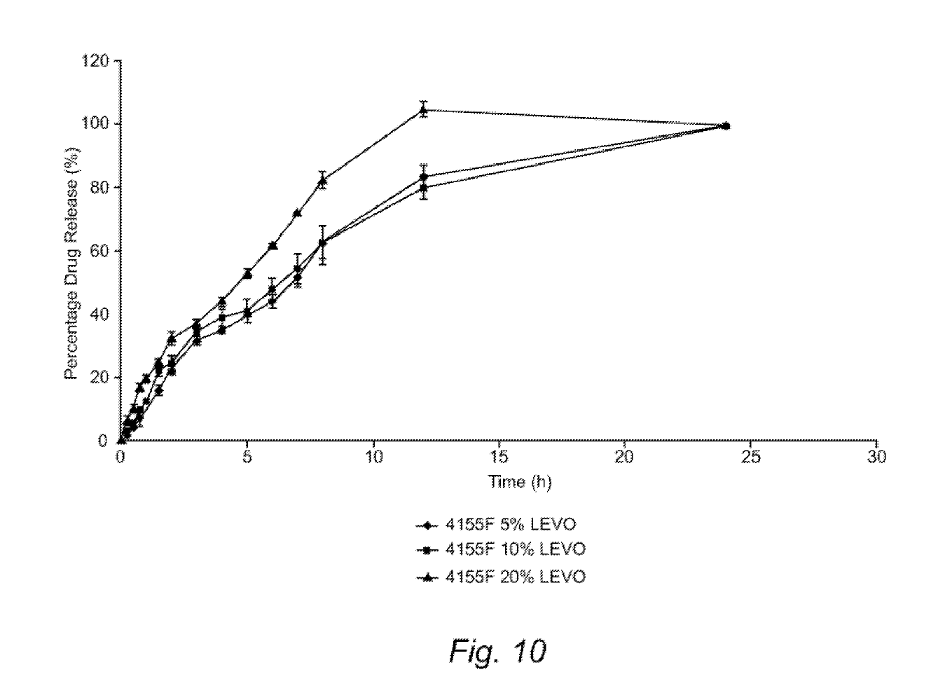

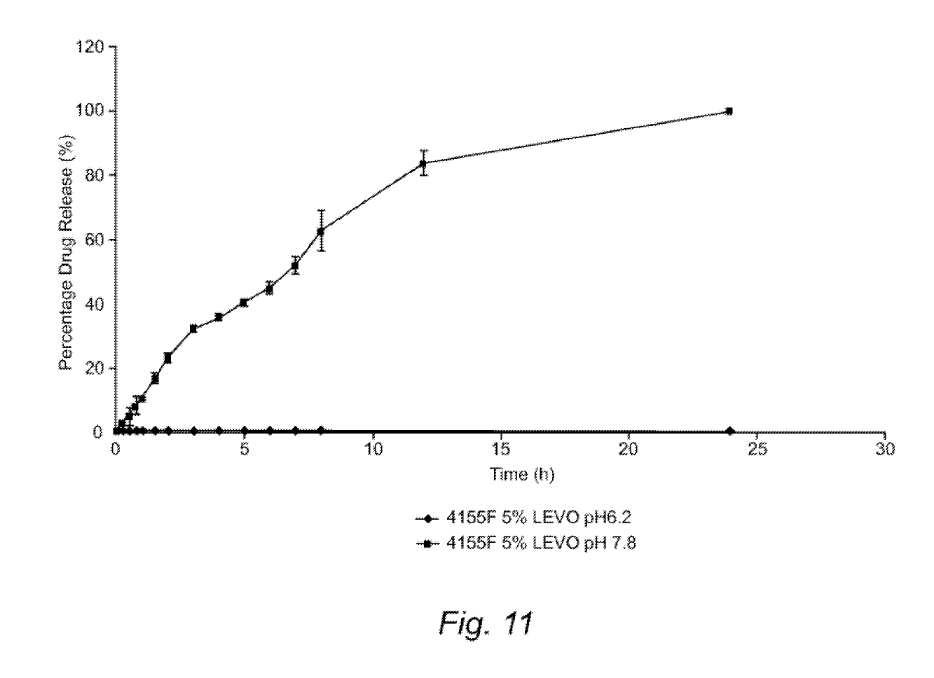

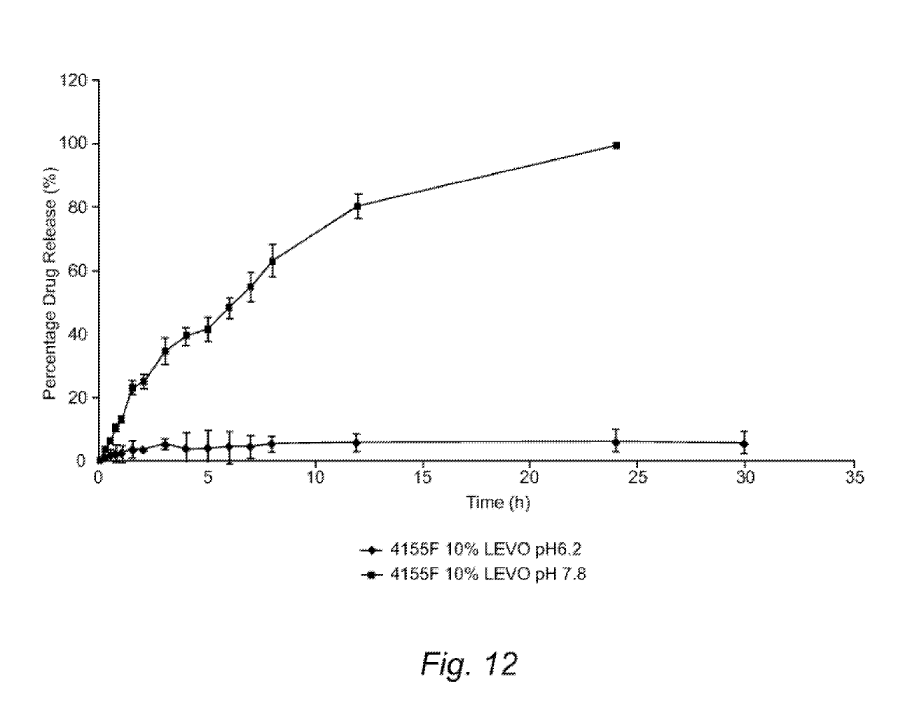

The polymeric matrix consisting of 4155F and levofloxacin has a much more controlled release of antimicrobial at pH 6.2 than L100. This is due to the fact that this polymer does not become soluble until the pH exceeds 7. This is very interesting as it will allow continuous elution of antimicrobial under ‘normal’ conditions. This should prevent bacterial adherence however should urease be produced (by P. mirabilis) and subsequently urea broken down to ammonia, the elevated pH increase will result in surface erosion and an increase in drug release rate. This is illustrated in FIGS. 9 to 12.

### Example 6

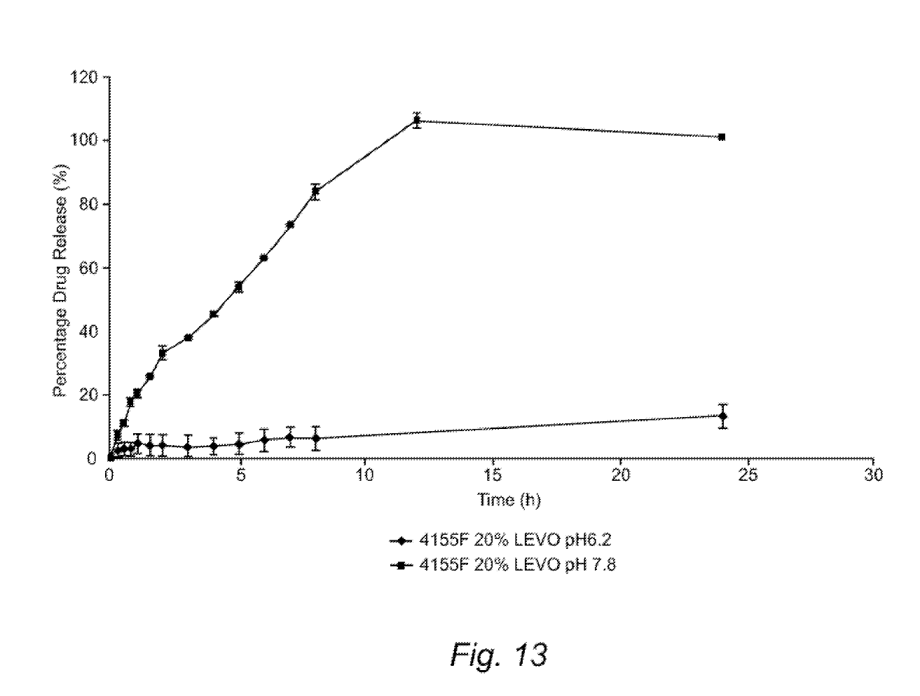

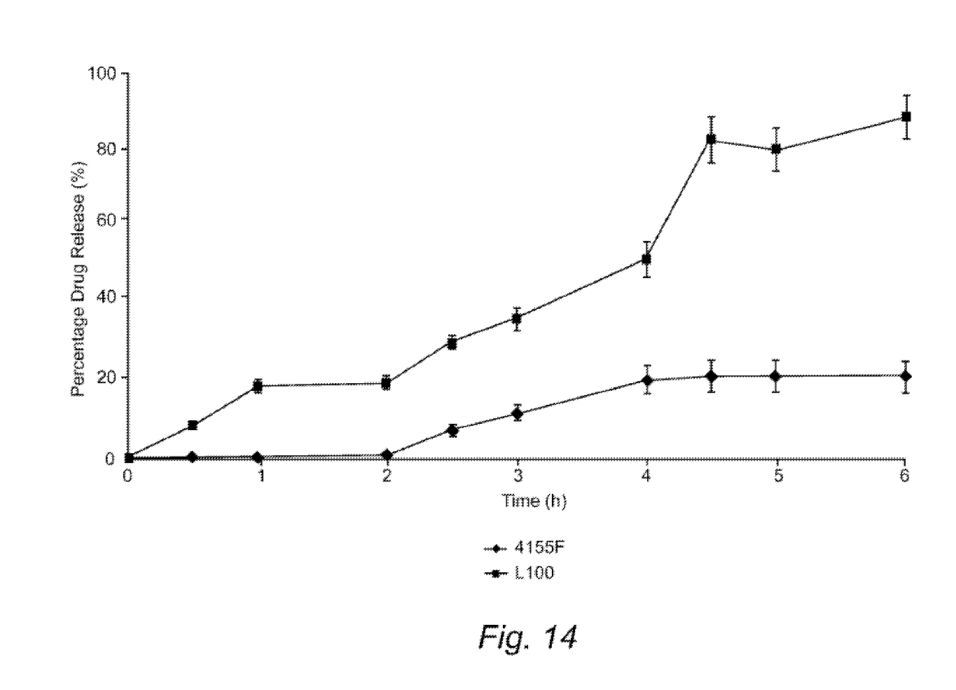

The devices of Example 5 were produced. The pH conditions surrounding the devices were maintained at pH 6.2 for 2 hours, and then adjusted to pH 7.8 for 2 hours before being adjusted back to pH 6.2 for 2 hours. FIGS. 13 and 14 illustrate the stop, start release profile of the devices of the present invention in response to changing pH conditions.

### Example 7

The devices of Example 5 were produced. These studies were conducted in pH 6.2 and pH 7.8 to assess the erosion (using mass change as an indicator) of the pH sensitive layers as a function of time and also to determine the effects of antimicrobial inclusion on this process. At pH 6.2 it is clear that the device comprising Eudragit® L100 maintains mass. There is a slight increase in mass due to water uptake during the study. Eudragit® L100 which begins to erode at pH values 6 shows almost complete loss after 24 hours.

The degradation of pH sensitive layer comprising Eudragit® L100 is extremely quick at 7.8 and this was expected. The pH sensitive layer comprising Eudragit® 4155F maintains mass at pH 7.8 but additionally increases mass due to water uptake.

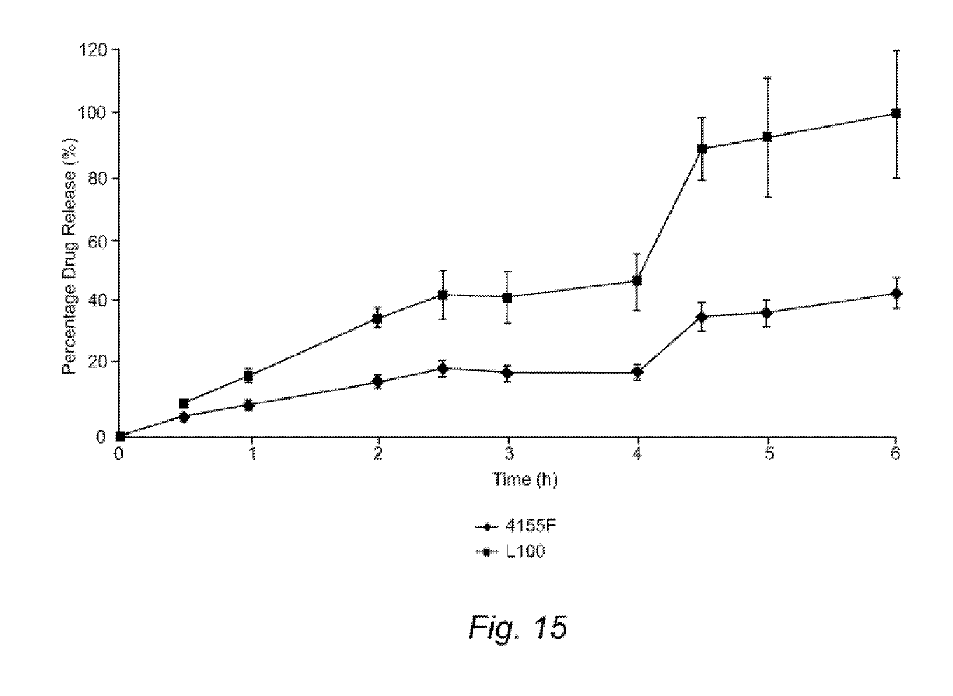

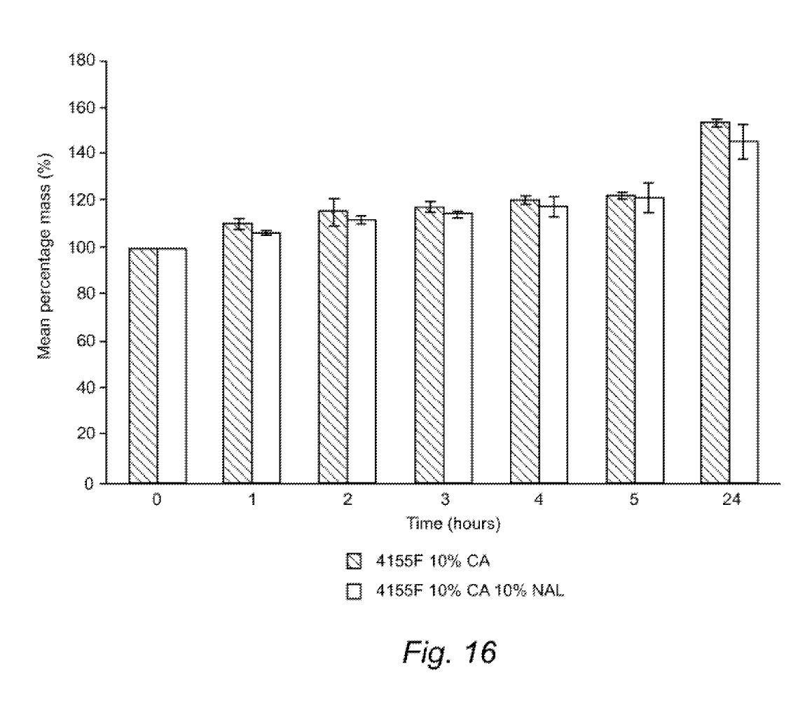

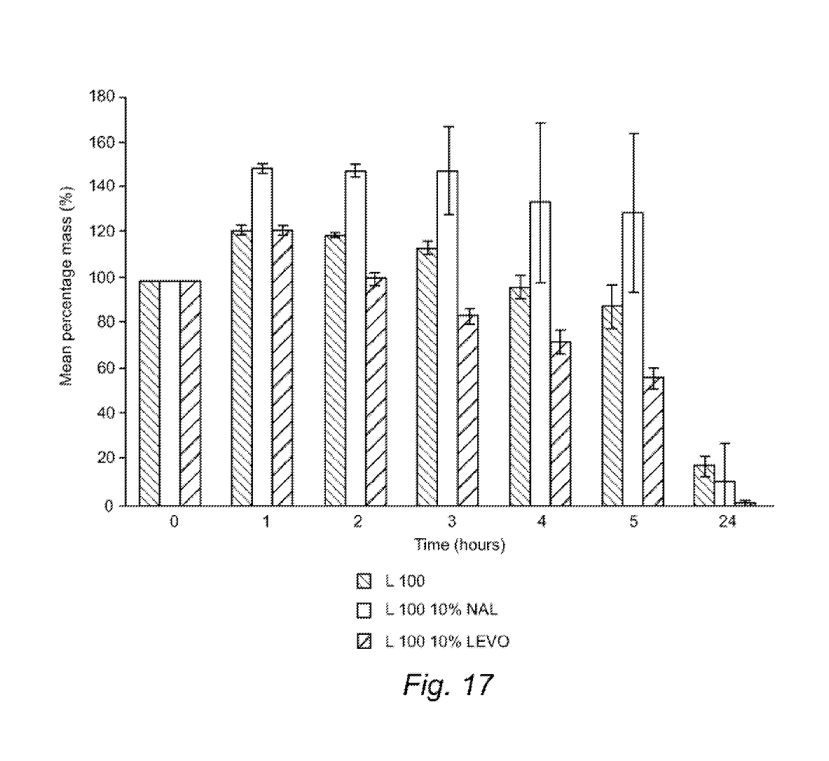

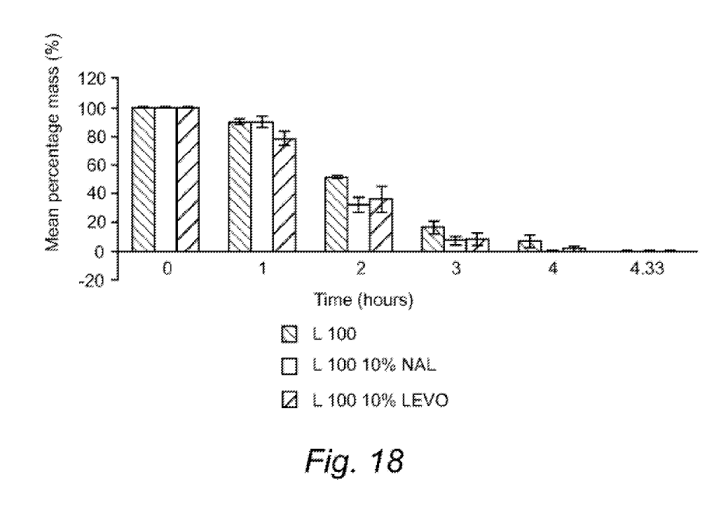

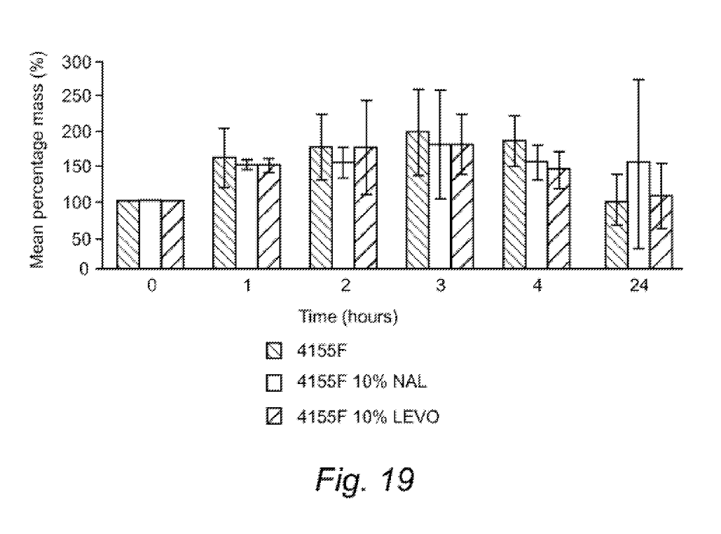

The erosion of the pH sensitive layers at pH 6.2 and 7.8 is illustrated in FIGS. 15 and 16.

### Example 8

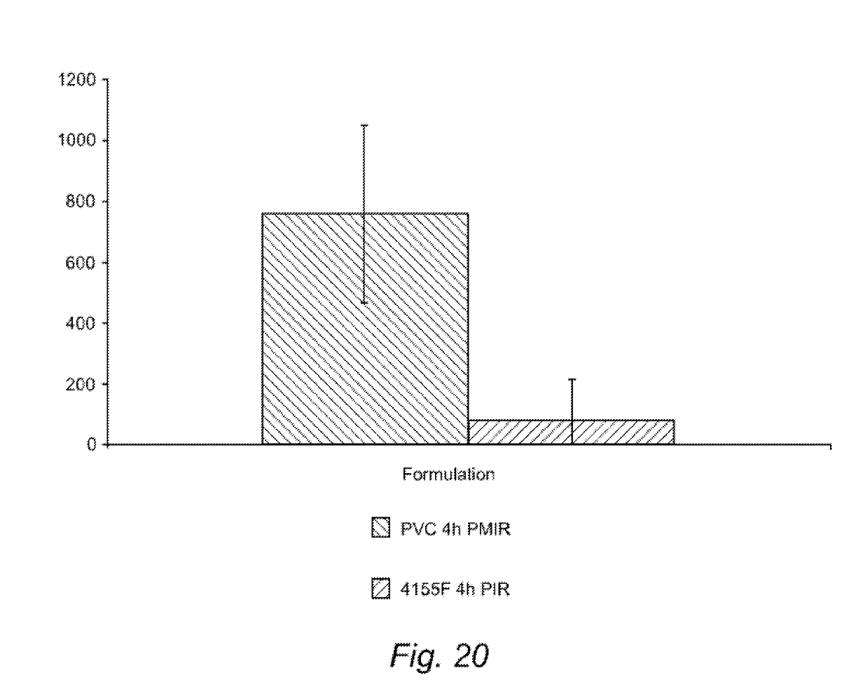

A first device was formed of PVC, and did not comprise a pH sensitive layer. A second device was formed of PVC, comprising a pH sensitive layer of Eudragit® 4155F. The two devices were immersed in artificial urine for 4 hours. The bacterial adherence of the two devices was then tested. The bacterial adherence to the first device was far greater than the bacterial adherence to the second device. The bacterial adherence was at least 8 times greater to the first device. This is illustrated in FIG. 20.

### Example 9

The tables below describe various specific embodiments of the multi-layered device (tube) of the invention described above in Tables 1-5. The layers below are numbered as they are in the tables above and are described in order from the interior-most layer defining the lumen to the exterior layer.

TABLE 9A
(Embodiment of FIG. 21A, corresponding to Table 1A)

| | General | |
|---|---|---|
| Layer | Description | Composition |
| 3 | Interior pH | Medical grade silicone |
| | sensitive layer | |
| | defining the | |
| | lumen | |
| 4 | Exterior | Erosion of layer will commence at pH values ≧7.0. |
| | structural layer | Layer comprising copolymer of Poly(methacylic acid-co- |
| | (non-pH | methyl methacrylate) 1:2 (Eudragit ® S100) |
| | sensitive) | |

TABLE 9B
(Embodiment of FIG. 21B, corresponding to Table 1B)

| | General | |
|---|---|---|
| Layer | Description | Properties and Composition |
| 7 | Exterior pH | Erosion of layer will commence at pH values ≧7.0. |
| | sensitive layer | Layer comprising copolymer of Poly(methacylic acid-co- |
| | | methyl methacrylate) 1:2 (Eudragit ® S100) |
| 6 | Interior | Medical grade silicone |
| | structural layer | |
| | (non-pH | |
| | sensitive) | |
| | defining the | |
| | lumen | |

TABLE 9C
(Embodiment of FIG. 22, corresponding to Table 2A)

| | General | |
|---|---|---|
| Layer | Description | Properties and Composition |
| 11 | Interior pH | Erosion of layer will commence at pH values ≧7.0. |
| | sensitive layer | Layer comprising copolymer of Poly(methacylic acid-co- |
| | defining the | methyl methacrylate) 1:2 (Eudragit ® S100) |
| | lumen | |
| 12 | Structural layer | Medical grade silicone |
| | (non-pH | |
| | sensitive) | |
| 13 | Exterior pH | Erosion of layer will commence at pH values ≧6.8. |
| | sensitive layer | Layer comprising partially esterified derivative of |
| | | hydroxypropyl methylcellulose containing 12% acetyl and |
| | | 7% succinoyl content, e.g. HPMC-AS HF grade (Shin-Etsu) |
| | | Alternatively, Erosion of layer will commence at pH values ≧7.0. |
| | | Layer comprising copolymer of Poly(methacylic acid- |
| | | co-methyl methacrylate) 1:2 (Eudragit S100) |

TABLE 9D
(Embodiment of FIG. 22, corresponding to Table 2B)

| | General | |
|---|---|---|
| Layer | Description | Properties and Composition |
| 11 | Interior pH | Erosion of layer will commence at pH values ≧7.0. |
| | sensitive layer | Layer comprising copolymer of Poly(methacylic acid-co- |
| | defining the | methyl methacrylate) 1:2 (Eudragit ® S100) |
| | lumen | |
| 12 | Structural layer | Medical grade silicone |
| | (non-pH | |
| | sensitive) | |
| 13 | Exterior pH | Erosion of layer will commence at pH values ≧6.0. |
| | sensitive layer | Layer comprising copolymer of Poly(methacylic acid-co- |
| | | methyl methacrylate) 1:1 (Eudragit L100). |

TABLE 9E
(Embodiment of FIG. 22, corresponding to Table 2C)

| | General | |
|---|---|---|
| Layer | Description | Properties and Composition |
| 11 | Interior pH | Erosion of layer will commence at pH values ≧6.0. |
| | sensitive layer | Layer comprising copolymer of Poly(methacylic acid-co- |
| | defining the | methyl methacrylate) 1:1 (Eudragit ® L100). |
| | lumen | |
| 12 | Structural layer | Medical grade silicone |
| | (non-pH | |
| | sensitive) | |
| 13 | Exterior pH | Erosion of layer will commence at pH values ≧6.8. |
| | sensitive layer | Layer comprising partially esterified derivative of |
| | | hydroxypropyl methylcellulose containing 12% acetyl and |
| | | 7% succinoyl content, e.g. HPMC-AS HF grade (Shin-Etsu) |

TABLE 9F
(Embodiment of FIG. 22, corresponding to Table 2D)

| | General | |
|---|---|---|
| Layer | Description | Properties and Composition |
| 11 | Interior pH | Erosion of layer will commence at pH values ≧6.0. |
| | sensitive layer | Layer comprising copolymer of Poly(methacylic acid-co- |
| | defining the | methyl methacrylate) 1:1 (Eudragit L100). |
| | lumen | |
| 12 | Structural layer | Medical grade silicone |
| | (non-pH | |
| | sensitive) | |
| 13 | Exterior pH | Erosion of layer will commence at pH values ≧6.0. |
| | sensitive layer | Layer comprising partially esterified derivative of |
| | | hydroxypropyl methylcellulose containing 9% acetyl and |
| | | 11% succinoyl content. Example being HPMC-AS MF grade |
| | | (Shin-Etsu) |

TABLE 9G
(Embodiment of FIG. 23A, corresponding to Table 3A)

| | General | |
|---|---|---|
| Layer | Description | Properties and Composition |
| 16 | Interior pH | Erosion of layer will commence at pH values ≧6.8. |
| | sensitive layer | Layer comprising partially esterified derivative of |
| | defining the | hydroxypropyl methylcellulose containing 12% acetyl and |
| | lumen | 7% succinoyl content, e.g. HPMC-AS HF grade (Shin-Etsu). |
| | | along with citric acid (5-30% wt). |
| 17 | Structural layer | Medical grade silicone |
| | (non-pH | |
| | sensitive) | |
| 18 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin in the range of 0.1-20% wt.. |
| 19 | Exterior pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer |
| | | contains also citric acid (5-30% wt.) and EDTA (5-20% wt) |
| | | that will chelate Mg and Ca salts and to buffer |
| | | microenvironment pH. |

TABLE 9H
(Embodiment of FIG. 23A, corresponding to Table 3B)

| | General | |
|---|---|---|
| Layer | Description | Properties and Composition |
| 16 | Interior pH | Erosion of layer will commence at pH values ≧7.0. |
| | sensitive layer | Layer comprising copolymer of Poly(methacylic acid-co- |
| | defining the | methyl methacrylate) 1:2 (Eudragit S100). This layer |
| | lumen | contains also citric acid (5-30% wt.) and EDTA (5-20% wt.) |
| | | that will chelate Mg and Ca salts and to buffer |
| | | microenvironment pH. |
| 17 | Structural layer | Medical grade silicone |
| | (non-pH | |
| | sensitive) | |
| 18 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin in the range of 0.1-20% wt. |
| 19 | Exterior pH | Erosion of layer will commence at pH values ≧6.0. |
| | sensitive layer | Layer comprising partially esterified derivative of |
| | | hydroxypropyl methylcellulose containing 9% acetyl and |
| | | 11% succinoyl content, e.g. HPMC-AS MF grade (Shin- |
| | | Etsu). Also included levofloxacin (0.1-20% wt.) and citric |
| | | acid (5-30% wt.). |

TABLE 9I
(Embodiment of FIG. 23B, corresponding to Table 4A)

| | General | |
|---|---|---|
| Layer | Description | Properties and Composition |
| 21 | Interior pH | Erosion of layer will commence at pH values ≧6.8. |
| | sensitive layer | Layer comprising partially esterified derivative of |
| | defining the | hydroxypropyl methylcellulose containing 12% acetyl and |
| | lumen | 7% succinoyl content. Example being HPMC-AS HF grade |
| | | (Shin-Etsu). This layer also contains citric acid (5-30% wt.), |
| | | levofloxacin (0.1-20%), and EDTA (5-20%). |
| 22 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin in the range 0.1-20%. |
| 23 | Structural layer | Medical grade silicone |
| | (non-pH | |
| | sensitive) | |
| 24 | Exterior pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20% wt.) and citric acid (5-15% |
| | | wt). |

TABLE 9J
(Embodiment of FIG. 23B, corresponding to Table 4B)

| | General | |
|---|---|---|
| Layer | Description | Properties and Composition |
| 21 | Interior pH | Erosion of layer will commence at pH values ≧6.0. |
| | sensitive layer | Layer comprising copolymer of Poly(methacylic acid-co- |
| | defining the | methyl methacrylate) 1:1 (Eudragit L100). Also included |
| | lumen | active agent (e.g., levofloxacin) at 0.1-20% wt. loading. |
| 22 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20% wt.), citric acid (5-15% wt.) |
| | | and EDTA (5-20% wt.)to control release of active at elevate |
| | | pH values, chelate Ca and Mg metal ions in urine. |
| 23 | Structural layer | Medical grade silicone |
| | (non-pH | |
| | sensitive) | |
| 24 | Exterior pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20% wt.), citric acid (5-15% wt.) |
| | | and EDTA (5-20%) to control release of active at elevate pH |
| | | values, chelate Ca and Mg metal ions in urine. |

TABLE 9K
(Embodiment of FIG. 24, corresponding to Table 5A)

| | General | |
|---|---|---|
| Layer | Description | Properties and Composition |
| 30 | Interior pH | Erosion of layer will commence at pH values ≧7.0. |
| | sensitive layer | Erosion of layer will commence at pH values ≧7.0. |
| | defining the | Layer comprising copolymer of Poly(methacylic acid-co- |
| | lumen | methyl methacrylate) 1:2 (Eudragit S100). This layer also |
| | | includes levofloxacin (0.1-20% wt.), citric acid (5-15% qt.) |
| | | and EDTA (5-20% wt.)to control release of active at elevate |
| | | pH values, chelate Ca and Mg metal ions in urine. |
| 29 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20% wt.), |
| 28 | Structural layer | Medical grade silicone |
| | (non-pH | |
| | sensitive) | |
| 27 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20% wt). |
| 26 | Exterior pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20%), citric acid (5-15% wt.) and |
| | | EDTA (5-20% wt.) to control release of active at elevate pH |
| | | values, chelate Ca and Mg metal ions in urine. |

TABLE 9L
(Embodiment of FIG. 24, corresponding to Table 5B)

| | General | |
|---|---|---|
| Layer | Description | Properties and Composition |
| 30 | Interior pH | Erosion of layer will commence at pH values ≧6.0. |
| | sensitive layer | Layer comprising copolymer of Poly(methacylic acid-co- |
| | defining the | methyl methacrylate) 1:1 (Eudragit L100). This layer also |
| | lumen | includes levofloxacin (0.1-20% wt.) |
| 29 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). This layer also |
| | | includes levofloxacin (0.1-20% wt.), citric acid (5-15% wt.) |
| | | and EDTA (5-20% wt.) to control release of active at |
| | | elevated pH values, chelate Ca and Mg metal ions in urine. |
| 28 | Structural layer | Medical grade silicone |
| | (non-pH | |
| | sensitive) | |
| 27 | Intermediate pH | Erosion of layer will commence at pH values ≧6.8. |
| | sensitive layer | Layer comprising partially esterified derivative of |
| | | hydroxypropyl methylcellulose containing 12% acetyl and |
| | | 7% succinoyl content. Example being HPMCAS-HF grade |
| | | (Shin-Etsu). This layer also contains citric acid (5-30% wt.). |
| 26 | Exterior pH | Erosion of layer will commence at pH values ≧6.8. |
| | sensitive layer | Layer comprising partially esterified derivative of |
| | | hydroxypropyl methylcellulose containing 12% acetyl and |
| | | 7% succinoyl content. Example being HPMCAS-HF grade |
| | | (Shin-Etsu). This layer also contains citric acid (5-30% wt.), |
| | | and levofloxacin (0.1-20% wt.). |

TABLE 9M
(Embodiment of FIG. 24, corresponding to Table 5C)

| | General | |
|---|---|---|
| Layer | Description | Properties and Composition |
| 30 | Interior pH | Erosion of layer will commence at pH values ≧7.0. |
| | sensitive layer | Layer comprising copolymer of Poly(methacylic acid-co- |
| | defining the | methyl methacrylate) 1:2 (Eudragit S100). This layer also |
| | lumen | includes levofloxacin in (0.1-20% wt.), citric acid (5-15% |
| | | wt.) and EDTA (5-20% wt.) to control release of active at |
| | | elevated pH values, chelate Ca and Mg metal ions in urine. |
| 29 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). |
| 28 | Structural layer | Medical grade silicone |
| | (non-pH | |
| | sensitive) | |
| 27 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). |
| 26 | Exterior pH | Erosion of layer will commence at pH values ≧6.0. |
| | sensitive layer | Layer comprising copolymer of Poly(methacylic acid-co- |
| | | methyl methacrylate) 1:1 (Eudragit L100). This layer also |
| | | includes levofloxacin (0.1-20% wt.) and EDTA (5-20%) to |
| | | chelate Ca and Mg metal ions in urine |

TABLE 9N
(Embodiment of FIG. 24, corresponding to Table 5D)

| | General | |
|---|---|---|
| Layer | Description | Properties and Composition |
| 30 | Interior pH | Erosion of layer will commence at pH values ≧6.0. |
| | sensitive layer | Layer comprising copolymer of Poly(methacylic acid-co- |
| | defining the | methyl methacrylate) 1:1 (Eudragit L100). This layer also |
| | lumen | includes levofloxacin (0.1-20% wt.) and EDTA (5-20%) to |
| | | chelate Ca and Mg metal ions in urine |
| 29 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). |
| 28 | Structural layer | Medical grade silicone. |
| | (non-pH | |
| | sensitive) | |
| 27 | Intermediate pH | Erosion of layer will commence at pH values ≧7.0. |
| | sensitive layer | Layer comprising copolymer of Poly(methacylic acid-co- |
| | | methyl methacrylate) 1:2 (Eudragit S100). |
| 26 | Exterior pH | Erosion of layer will commence at pH values ≧6.0. |
| | sensitive layer | Layer comprising copolymer of Poly(methacylic acid-co- |
| | | methyl methacrylate) 1:1 (Eudragit L100). This layer also |
| | | includes levofloxacin (0.1-20% wt.) and EDTA (5-20% wt.) |
| | | to chelate Ca and Mg metal ions in urine |

### Example 10

A multilayered device of the invention can be made by extrusion according to the method below or other methods known in the industry. This extrusion method is a method for preparing a device (catheter or stent) having at least one structural layer and one or more pH sensitive layers. A hot-melt extruder e.g. a Randcastle Taskmaster hot-melt extruder, is equipped with a multi-layer tube die to produce a range of multi-layer tube structures of different overall wall thicknesses, depending on the particular device application. Medical tubing products (e.g. urinary catheters and stents) are elastomeric in nature. i.e. they have comparatively low modulus at body temperature to allow for easy insertion into the body. They must also exhibit a certain degree of kink resistance (largely controlled by careful choice of wall thickness) that may otherwise impede body fluid drainage. The extruder temperature (typically about 65-135° C.), motor revolutions (typically about 60-90 RPM) and motor drive current (typically about 6-9 amps) are selected to provide adequate molten polymer flow and viscosity for each of the polymer melts used to make the respective layers. Powders, from which each of the layers is made, are blended prior to extrusion. The powders comprise one or more polymers, one or more functional excipients, one or more active agents and/or one or more pharmaceutical excipients as dictated by the intended compositions of respective layers. The melt extruder having multiple temperature zones can be set as needed according to the melting point of the composition in corresponding feed hoppers, for example, zone 1: 65° C., zone 2: 120° C., zone 3: 125° C., zone 4: 135° C., die temperature 135° C. One or more powder blends are placed in one or more feed hoppers that are located at the head of a horizontal screw such that the material is starve fed by a mass flow controller operated at a solids flow rate of sufficient to provide uniform layers, e.g. 0.5 to 10 kg/hr. A feed hopper will contain the materials forming the structural layer, and one or more feed hoppers will contain the materials forming each respective pH sensitive layers. The residence time of the material in the extruder can be about 0.5-5 minutes or as needed, depending upon screw speed, feed rate, performance or other such variables. The order of the layers can be varied simply by charging different compositions into the feed hoppers. The extrudate is cut into sections after exiting the die and allowed to cool.

The feed hoppers can be charged as follows for preparing the indicated devices (PSP denotes pH sensitive polymer composition; SLP denotes structural layer polymer composition):

| Hop- | Embodiment of: | | | | | |
|---|---|---|---|---|---|---|
| per | FIG. 21A | FIG. 21B | FIG. 22 | FIG. 23A | FIG. 23B | FIG. 24 |
| 1 | PSP | SLP | PSP | PSP | PSP | PSP |
| 2 | SLP | PSP | SLP | SLP | PSP | PSP |
| 3 | | | PSP | PSP | SLP | SLP |
| 4 | | | | PSP | PSP | PSP |
| 5 | | | | | | PSP |

The term “about” is intended to mean±10%, ±5%, ±2.5% or ±1% relative to a specified value, i.e. “about” 22% means 22±2.2%, 22±1.1%, 22±0.55% or 22±0.22%.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1. A device comprising a body structure having one or more surfaces wherein at least one of the surfaces comprises a pH sensitive layer comprising a linear polymer, wherein the water solubility of the linear polymer increases from a first water solubility to a second water solubility at a pH trigger and the linear polymer undergoes dissolution or erosion in an aqueous environment at the second water solubility.

2-13. (canceled)

14. The device of claim 1, wherein the device comprises more than three pH sensitive layers.

15. A method of forming a device comprising the steps of providing a structural layer and applying at least one pH sensitive layer thereto, said pH sensitive layer comprising a linear polymer wherein the water solubility of the linear polymer increases from a first water solubility to a second water solubility at a pH trigger.

16-19. (canceled)

20. A method of preventing or mitigating infection associated with a device implanted or inserted into the human or animal body comprising the step of implanting or inserting a device into the human or animal body, wherein said device is a device according to claim 1.

21-27. (canceled)

28. A device comprising an extended body structure comprising:
an extended structural layer having an inside surface and an outside surface, said inside surface defining an extended lumen, wherein the structural layer is substantially non-degradable or non-erodible at a pH of 2 to 10 and provides structural stability to the device;
at least one extended pH sensitive degradable layer attached to, in centric arrangement with, and coextensive with the structural layer and being capable of controlled erosion, dissolution or degradation at a pH in the range of ≧5 to ≧7, each degradable layer comprising a pH sensitive linear polymer that ionizes and dissolves in an aqueous environment of use when the pH of the environment moves away from an initial pH to a pH that is indicative of bacterial contamination, wherein the water solubility of the linear polymer increases from a first water solubility to a second water solubility at a pH trigger.

29. (canceled)

30. (canceled)

31. The device of claim 28 wherein plural pH sensitive degradable layers are present.

32-62. (canceled)

63. A multilayered catheter or stent comprising plural coextensive and centrically arranged layers, said layers defining a lumen, wherein:
a first coextensive layer is a structural layer that is substantially non-degradable or non-erodible under physiological conditions; and
at least one second coextensive layer is a pH sensitive layer comprising one or more pH sensitive linear polymers that ionize and dissolve, erode or degrade in an aqueous environment when the second coextensive layer is contaminated with bacteria.

64. The device according to claim 63 wherein the at least one second coextensive layer is in the interior of the first coextensive layer.

65. The device according to claim 63 wherein the at least one second coextensive layer is at the exterior of the first coextensive layer.

66. The device according to claim 63 wherein at least one second coextensive layer is in the interior of the first coextensive layer, and at least one second coextensive layer is at the exterior of the first coextensive layer.

67. The device according to claim 66 wherein two second coextensive layers are in the interior of the first coextensive layer, and one second coextensive layer is at the exterior of the first coextensive layer.

68. The device according to claim 67 wherein two interior second coextensive layers ionize and dissolve, erode or degrade at different pH values in an aqueous environment.

69. The device according to claim 67 wherein all second coextensive layers ionize and dissolve, erode or degrade at the same pH values in an aqueous environment

70. The device according to claim 66 wherein two second coextensive layers are at the exterior of the first coextensive layer, and one second coextensive layer is in the interior of the first coextensive layer.

71. The device according to claim 70 wherein two exterior second coextensive layers ionize and dissolve, erode or degrade at different pH values in an aqueous environment.

72. The device according to claim 66 wherein two second coextensive layers are in the interior of the first coextensive layer, and two second coextensive layers are at the exterior of the first coextensive layer.

73. The device according to claim 72 wherein two interior second coextensive layers ionize and dissolve, erode or degrade at different pH values in an aqueous environment.

74. The device according to claim 73 wherein two exterior second coextensive layers ionize and dissolve, erode or degrade at the same pH values in an aqueous environment.

75. The device according to claim 72 wherein two exterior second coextensive layers ionize and dissolve, erode or degrade at different pH values in an aqueous environment.

76. The device according to claim 75 wherein two interior second coextensive layers ionize and dissolve, erode or degrade at the same pH values in an aqueous environment.

77. The device according to claim 72 wherein all second coextensive layers ionize and dissolve, erode or degrade at the same pH values in an aqueous environment.

78. The device according to claim 63 wherein the bacterial contamination is caused by Escherichia coli, Lactobacillus bacterium, Proteus bacterium, other such bacteria known to be capable of contaminating the urinary tract.

79. The device according to claim 63 wherein the pH sensitive layer excludes a cross-linked pH sensitive polymer.

80. The device according to claim 63 wherein the pH sensitive layer comprises one or more linear pH sensitive polymers.

81. The device according to claim 63 wherein the structural layer comprises one or more of silicone, latex, (poly (vinyl chloride)), polyurethane, ethylene-vinylacetate copolymer, polyethylene, polypropylene, polyester, polystyrene, nylon.

82. The device according to claim 63 wherein the pH sensitive polymer is a cellulose-based linear polymer of the Formula I, wherein: a) at least one R is H, at least one R is —CH₃, at least one R is —CH₂CH(OH)CH₃, at least one R is —COCH₃, and at least one R is —COCH₂CH₂COOH, optionally wherein at least one R is —CH₂CH(OCOCH₂CH₂COOH)CH₃ or at least one R is —CH₂CH(OCOCH₃)CH₃; b) at least one R is H, at least one R is —CH₃, at least one R is —CH₂CH(OH)CH₃, and at least one R is —CO(C₆H₄)CO₂H, optionally wherein at least one R is —CH₂CH(OCO(C₆H₄)CO₂H)CH₃; c) at least one R is H, at least one R is —COCH₃, and at least one R is —CO(C₆H₄)CO₂H; d) at least one R is H, at least one R is —COCH₃, and at least one R is —COCH₂CH₂CH₃; or e) at least one R is H, at least one R is —COCH₃, and at least one R is —CO(C₆H₃)(CO₂H)₂.

83. The device according to claim 63 wherein the pH trigger is ≧5, ≧5.5, ≧6, ≧6.2, ≧6.5, ≧6.8 or ≧7.

84. The device according to claim 63 wherein the linear pH sensitive polymer is selected from the group consisting of: a) hydroxypropyl methylcellulose acetate succinate having a pH trigger of ≧5.5, ≧6 or ≧6.8; b) hydroxypropyl methylcellulose phthalate having a pH trigger of ≧5 or ≧5.5; c) cellulose acetate trimellitate having a pH trigger of ≧5; d) cellulose acetate phthalate having a pH trigger of ≧6.2; or e) cellulose acetate butyrate having a pH trigger of ≧6.

85. The device according to claim 83 wherein the pH sensitive polymer having a pH trigger of about 7 is selected from the group consisting of copolymers of methacrylic acid and methyl methacrylate containing at least 2 methyl methacrylate units per methacrylic acid unit, partially esterified derivatives of hydroxypropyl methylcellulose containing at >/=12% acetyl content and </=7% succinoyl content.

86. The device according to claim 83 wherein the pH sensitive polymer having a pH trigger of about 6 is selected from the group consisting of partially esterified derivatives of hydroxypropyl methylcellulose containing at >/=9% acetyl content and </=11% succinoyl content, copolymers of methacrylic acid and methyl methacrylate containing at least 1 methyl methacrylate unit per methacrylic acid unit, cellulose acetate phthalate, cellulose acetate trimellitate, copolymers of ethylacrylate and methacrylic acid at a ratio of 1:1, wherein the layer further comprises one or more organic acids.
